(19) United States
(12) Patent Application Publication
(10) Pub. No.: US 2013/0023767 A1
(43) Pub. Date: Jan. 24, 2013

(54) LOW-COST, HIGH FIDELITY ULTRASOUND SYSTEM

(71) Applicant: Richard J. Mammone, Warren, NJ (US)

(72) Inventor: Richard J. Mammone, Warren, NJ (US)

(21) Appl. No.: 13/471,448

(22) Filed: May 14, 2012

Related U.S. Application Data
(60) Provisional application No. 61/485,524, filed on May 12, 2011.
(60) Provisional application No. 61/497,398, filed on Jun. 15, 2011.

(51) Int. Cl. A61B 8/08; A61B 8/14

(52) U.S. Cl. 600/440; 600/443; 600/442; 600/447

(57) ABSTRACT

A system and method for performing an ultrasound may include generating a set of continuous tone signals for injection into an object. A corresponding set of reflected tone signals in the frequency domain may be received The set of reflected tone signals may be converted from the frequency domain to the time domain to create a set of time domain signals. At least one region of interest may be identified from the set of time domain signals. A window may be defined around the identified region of interest in the set of time domain signals. The windowed time domain signals may be converted from the time domain to the frequency domain to create a set of windowed frequency domain signals. At least one characteristic parameter may be calculated from the set of windowed frequency domain signals. Information may be output based on the calculate at least one characteristic parameter.

### RELATED APPLICATIONS

This application claims priority to co-pending U.S. Provisional Patent Application Ser. No. 61/485,524 entitled, “CONTINUOUS WAVE ULTRASOUND FOR THE MEASUREMENT OF FREQUENCY VARYING ECHOGENITY,” filed May 12, 2011 and U.S. Provisional Patent Application Ser. No. 61/497,398 entitled, “PROBE NAVIGATION GUIDANCE SYSTEM FOR QUICK AND CONSISTENT WHOLE BREAST ULTRASOUND SCANS,” filed Jun. 15, 2011; the contents of which are incorporate herein by reference in their entirety.

### BACKGROUND

Cancer is a leading cause of death in the world today. Breast cancer, in particular, is a major cause of death among women. There is an unmet need for an accurate low-cost cancer diagnostic system that can be deployed in point-of-care facilities on a global basis. As well understood in the art, early detection and diagnosis is essential for reducing the mortality, and improving the clinical curative rate and life quality of the cancer patients. Ultrasonography has been a valuable method of identifying cancer and other tissue features due to its quick imaging and high reproducibility for breast cancer detection. High frequency, low-cost broadband ultrasound transducers that employ well-known technologies, such as PZT transducers and newer technologies, such as capacitive micromachined ultrasonic transducers (CMUTs) and other techniques are becoming widely available. These high-fidelity transducers can be easily connected to a personal computer, laptop or mobile phone that can process and display the measured signals for an ultrasound technician or physician. The resulting system is portable, but still have the shortcomings as conventional ultrasound systems and unable to compete with more costly, high-fidelity ultrasound imaging systems or be accurate enough to ultimately compete with mammography as the standard of care.

In the standard process for breast cancer screening, for example, an area is typically first identified as a region of interest (ROI) if it appears as a lesion or a cluster of small calcifications called micro-calcifications. The question as to whether the ROI includes tissue that is malignant or benign can be answered by using ultrasound to examine the acoustic impedance of the lesion to see how “hard” and regularly shaped it is. Cancerous lesions are generally harder and more irregularly shaped than normal tissue. The ability for an ultrasound imaging system to measure small variations in acoustic impedance is called its contrast resolution.

Conventional ultrasound imaging systems provide an image that indicates the change in acoustic impedance ΔZ=Zi−Zi−1 between consecutive layers of the different tissues encountered by an ultrasound pulse as it travels through a part of the body. The acoustic impedance profile is visualized indirectly by displaying the measured reflection coefficient Ri=ΔZi/(Zi+Zi−1) or transmission coefficient Ti=1−Ri of the pulse as it changes with time/distance along its path. Generally, the reflection coefficient is observed using various display formats such A-scans, B-scans, 3D displays, etc., to help visualize the structure of the body traveled by the injected ultrasound pulse/pulses. The structural information, however, is not highly indicative of the tissue type, such as cancerous or benign tissue. There is a need to differentiate tissue type, such as cancer or otherwise, non-invasively. However, because of the inherent limitations of conventional ultrasound systems, such non-invasive determination is either limited or not possible.

Contrast resolution refers to the ability to distinguish echogenicity differences between neighboring soft tissue regions. The higher the contrast resolution of a system the more likely the operator can see subtle differences in tissue characteristics that might indicate the early stages of cancer. Another feature used as an indicator of cancer is micro-calcifications that may appear in ducts or lobules of a cancer patient. The size of the individual calcified particles, the regularity of the shape, and the size of the cluster have been found to give early indication of breast cancer. The spatial resolution of a ultrasound system must be very high to allow the small microcalcifications to be seen. Currently, only expensive ultrasound imaging systems with the highest spatial resolution can provide images with enough detail to see micro-calcifications that are indicative of the presence of cancer. Thus, another property desired for high fidelity ultrasound system is high spatial resolution. In addition, the accuracy of most indicators of tissue characteristics, such as attenuation, speed of sound, acoustic impedance, and harmonic content, depends on measuring a Fourier transform of the signal with a high frequency resolution, which is generally not possible with conventional ultrasound systems.

Breast micro-calcifications are calcium deposits within the breast tissue. The micro-calcifications tend to appear as white spots or flecks on a mammogram and are usually so small that they cannot be felt by a human hand. Non-invasive cancers, known as ductal carcinoma in situ (DCIS), confined to the ducts of the breast have been indicated by certain constellations of small calcifications called micro-calcifications. The size, morphology, and distribution of micro-calcifications are important indicators in the mammographic screening for and diagnosis of various carcinomas in the breast. Micro-calcifications are typically 50 to 500 microns in size. Although breast micro-calcifications are usually noncancerous (benign), certain patterns of micro-calcifications, such as tight clusters with irregular shapes, may indicate breast cancer. It should be understood that other cancers and non-cancers may have other identifiable characteristics.

Although x-ray mammography is currently the only accepted method for detecting micro-calcifications, its efficacy in this regard can be reduced in the presence of dense parenchyma usually found in younger women. It has been estimated that 35% to 45% of screen detected non-palpable breast cancers are discovered because of the presence of clustered micro-classification on mammography. It is also estimated that 30% to 50% of breast cancers have micro-calcifications clusters associated with the detected lesion or mass.

Conventional ultrasound scanners cannot reliably detect micro-calcifications in the size range of clinical interest due to spatial resolution limitations and speckle noise. However, the use of real-time, high frequency (above 7.5 MHz) transducers have been shown to be able to detect micro-calcifications as highly reflective specs (micro-calcifications) within a lower refection area (a lesion). These transducers tend to appear larger than the actual pathological size and do not attenuate. Using 7.5-10 MHz real-time ultrasound equipment, ultrasound abnormalities corresponding to clustered micro-calcifications can be identified in 60-76% cases. The use of high frequency ultrasound probes (HFUS) operating above 7.5 MHz have axial and lateral resolutions above 0.1 to 0.5 mm or 100 to 500 microns. The use of 13 MHz axial resolution of 0.118 mm can detect 150 micron calcifications. Thus, high frequency operation is imperative for accurate early diagnosis of breast cancer.

Despite being able to detect micro-calcifications, an inherent problem that exists with conventional ultrasound transducers is that resolution is too inaccurate for a determination of location of the cancer. As understood in the art, in vitro (outside the body) measurement techniques exist. However, in vivo (inside the body) techniques using conventional ultrasound systems are not performed using ultrasound because of distortion and other technical issues. As an example, the use of time-based, pulse sensing techniques of conventional ultrasound systems produce images that are inherently inaccurate for micro-calcification sizes at least in part due to the relatively long time duration of the pulses used to illuminate the micro-calcifications.

Another difficulty in using ultrasound to detect micro-calcifications is speckle noise, which can both mask a true micro-calcification or appear as a false micro-calcification. Speckle is created by the complex interference of ultrasound echoes made by reflectors spaced closer than the resolution limits of the machine. The issue of speckle can be reduced by using higher frequency imaging systems that enhances the spatial resolution limit of the system and lowers the resolution cell, thus, increasing the operating frequency of the ultrasound improves spatial resolution in both lateral and axial directions and reduces the effects of speckle noise. The trade-offs in designing a higher frequency ultrasound imaging system include (i) increased cost and (ii) a greater attenuation of the received energy at higher frequencies since signal-to-noise ratio (SNR) decreases exponentially with depth. The attenuation trade-off is usually resolved by either limiting the depth, which can be visualized due to the limited energy available per pulse, or by using coded pulses and/or chirped pulses that allow longer pulses and, thus, more energy, which requires more bandwidth. Thus, the increased attenuation due to increasing the operating frequency can be offset by increasing the bandwidth of the system and preserving the depth penetration. This increase in bandwidth also increases axial resolution. Of course, the increased bandwidth results in higher cost of the ultrasound system.

In summary, the use of high frequency wideband ultrasound probes is preferred to visualize micro-calcifications while providing visualization of subtle tissue variations and maintaining maximal penetration depth. However, the operation of the ultrasound system at high frequencies with a large bandwidth imposes additional system requirements on the electronics and signal processing algorithms used in standard ultrasound imaging systems increase cost by a large margin.

The choice of frequency is a trade-off between spatial resolution of the image and imaging depth: lower frequencies produce less resolution, but image deeper into the body. Higher frequency sound waves have a smaller wavelength and, thus, are capable of reflecting or scattering from smaller structures. Higher frequency sound waves also have a larger attenuation coefficient and, thus, are more readily absorbed in tissue, limiting the depth of penetration of the sound wave into the body. Though ultrasound resolution is affected by several factors, imaging frequency has the most direct impact. Ultrasound resolution improves in direct proportion to imaging frequency. In a typical 5-10 MHz system, the resolution cell measures roughly 0.7×0.35 mm. The result is that anatomical structures smaller than 1 mm are likely to be missed. Contrast resolution allows the ultrasonographer to distinguish between subtle solid lesions from surrounding normal fatty or glandular tissues. Contrast resolution depends on the SNR of the measured signal. Contrast resolution also depends on spatial resolution, suppression of side lobes, and is better for higher bandwidth and higher frequency systems.

### SUMMARY

To overcome the shortcomings of conventional ultrasound imaging systems and devices, the principles of the present invention provide for a low-cost ultrasound imaging system with high spatial, high frequency, and contrast resolution that can be used to improve diagnoses of cancers and other tissue features. In accordance with the principles of the present invention, a multi-domain process may be utilized in performing measurements. In one embodiment, the multi-domain measurement process may include utilizing a set of continuous tones in the frequency domain, receiving reflected tones in the frequency domain via a transducer, converting the set of tones into the time domain, identifying reflection features in the time domain that represent a region of interest (e.g., a selected region of tissue that may represent cancer or other pathology), creating a window around the identified reflection features in the time domain, converting the samples within the window back into the frequency domain to form a high fidelity signal, generating tissue characteristic parameters from the time or frequency domain version of the signal, and determining a probability that cancer or other pathology of the tissue exists within the region of interest based on the tissue characteristic parameters. In one embodiment, the system may operate at high frequencies (above 7.5 MHz) with a large bandwidth (percent bandwidth of 60%-90%, where the percent bandwidth is the absolute bandwidth divided by the carrier frequency) using low cost electronics and software suitable to be run on a small computing device, such as a personal computer or mobile device.

Furthermore, the principles of the present invention operate at higher frequencies than conventional pulse echo ultrasound systems due to the longer depth penetration that results from higher SNR. The contrast resolution is also enhanced due to the higher SNR, thereby providing improved ability to detect small variations in tissue characteristics. In addition, contrast resolution is improved by providing frequency resolution frequency domain representation of the tissue in a selected region of interest. The higher frequency provides for higher lateral resolution. The higher SNR also provides improved contrast resolution, which allows for improved classification of tissue characteristics. In addition, compensation for variations in the speed of sound and attenuation of the ultrasound signal in the various tissues along the scan line may be made for the scan that is being measured and/or displayed.

One embodiment of an ultrasound system may include a processing unit, a transducer, and a signal generator in communication with the computing device and transducer. A receiver may be in communication with the processing unit and transducer. The computing device may be configured to cause the signal generator to generate a set of ultrasound signals ranging from a first frequency to a second frequency and output the set of ultrasound signals via the transducer into an object. The receiver may be configured to receive a set of reflected ultrasound signals via the transducer, the processing unit may further be configured to (i) calculate a set of reflection signals that are integrated over a dwell time, where the set of reflection signals may be stored as a function of frequency, (ii) subsample the set of reflection signals, (iii) convert the subsampled set of reflection signals into the time domain, (iv) identify a region of interest in the object based on the subsampled set of reflection signals in the time domain, (v) convert the subsampled set of reflection signals from the time domain into the frequency domain, (vi) combine, in the frequency domain, the converted set of subsampled reflection signals, (vii) determine at least one characteristic parameter associated with the identified region of interest, and (viii) output information based on the determined at least one characteristic parameter to a user of the ultrasound system.

One method for performing an ultrasound may include generating a set of continuous tone signals in the frequency domain for injection into an object. A corresponding set of reflected tone signals in the frequency domain may be received The set of reflected tone signals may be converted from the frequency domain to the time domain to create a set of time domain signals. At least one region of interest may be identified from the set of time domain signals. A window may be defined around the identified region of interest in the set of time domain signals. The windowed time domain signals may be converted from the time domain to the frequency domain to create a set of windowed frequency domain signals. At least one characteristic parameter may be calculated from the set of windowed frequency domain signals. Information may be output based on the calculate at least one characteristic parameter to a user of the ultrasound system.

One method for performing an ultrasound may include generating a set of continuous tone signals in the frequency domain for injection into an object. A corresponding set of reflected tone signals may be received in the frequency domain. The set of reflected tone signals may be converted from the frequency domain to the time domain to create a set of time domain signals. At least one region of interest may be identified from the set of time domain signals. The time domain signals may be converted from the time domain to the frequency domain to create a set of frequency domain signals. At least one characteristic parameter may be converted from the set of frequency domain signals. The set of frequency domain signals may be compensated by the characteristic parameter(s) to create a compensated set of frequency domain signals. The compensated set of frequency domain signals may be displayed.

A structure for supporting an ultrasound probe for imaging an anatomical region of a patient may include a first member being semi-rigid and having a pre-determined geometrical shape. An array of transducers may be supported by and positioned relative to the first member. A bladder may have a size and shape that conforms to being positioned between the first member and the anatomical region of the patient. A securing member may be utilized that causes the first member, array of transducers, and bladder to maintain position relative to the anatomical region of the patient.

### BRIEF DESCRIPTION OF THE DRAWINGS

Illustrative embodiments of the present invention are described in detail below with reference to the attached drawing figures, which are incorporated by reference herein and wherein:

FIG. 1 is an illustration of an illustrative ultrasound probe that utilizes the principles of the present invention;

FIG. 2 is a graph of an illustrative continuous wave signal that may be used by an ultrasound system shown in both the frequency and time domains;

FIG. 3 is a graph showing an illustrative total bandwidth for a continuous wave signal for use in an ultrasound system in accordance with the principles of the present invention;

FIGS. 4A and 4B (collectively FIG. 4) are real and imaginary graphs of illustrative reflection coefficient signals or patterns that may be observed for measured reflection coefficients over a range of frequencies for a single reflector;

FIG. 5 is a graph showing an illustrative sinc (sin(x)/x) function signal in the time domain that defines a tone signal and that may be utilized to define a window that includes a measurement feature for further processing;

FIG. 6 is a graph that shows multiple reflection signals in the time domain from using an ultrasound system with continuous tones in accordance with the principles of the present invention;

FIG. 7 is a chart that shows typical variation in acoustic impedance found between cancerous tissues and benign tissues;

FIG. 8 is a chart that illustrates a typical color code used to help visualize acoustic impedance to better enable an ultrasound technician distinguish between cancerous and non-cancerous tissues;

FIG. 9 is a block diagram of an illustrative ultrasound signal generator and ultrasound probe of FIG. 1 that may be utilized in an ultrasound system in accordance with the principles of the present invention;

FIG. 10 is a block diagram of an illustrative ultrasound system that may utilize the principles of the present invention in performing low-cost ultrasound imaging;

FIG. 11 is a flow diagram of an illustrative process that may be utilized by the system of FIG. 10 in performing ultrasound imaging and analysis for determining whether cancer exists in imaged tissue;

FIG. 12 is an illustration of an illustrative ultrasound system that may utilize the principles of the present invention to image and detect cancer in tissue of a patient;

FIG. 13 is a graph showing an illustrative RF transmission line spectrum responsive to a non-linear tissue when excited by an ultrasound tone or signal;

FIG. 14A-14C (collectively FIG. 14) are graphs showing a time sequence that illustrates the generation of harmonic frequencies;

FIG. 15 is a schematic that depicts relative intensities and frequency changes of harmonic ultrasound beams and fundamental transmitted waves with increasing depth in tissues;

FIG. 16 is an illustration of an illustrative ultrasound system showing processing of reflected ultrasound signals to determine whether cancer exists in tissue being imaged by the ultrasound system;

FIGS. 17A-17E are graphs of illustrative signals that are used in performing an ultrasound scan and determining a tissue characteristic, in this case an attenuation constant α₁, to determine the type of tissue being scanned;

FIG. 18 is a block diagram of an illustrative process for processing reflected ultrasound signals in accordance with the principles of the present invention;

FIG. 19A is an illustration of components of an illustrative structure, in this case a bra, including a bra cup and an illustrative transducer array that may be used to perform ultrasound imaging in accordance with the principles of the present invention;

FIG. 19B is another illustration of components of the illustrative structure of FIG. 19A, in this case the bra, that utilizes an illustrative bladder that may be utilized to assist in performing ultrasound imaging in accordance with the principles of the present invention; and

FIG. 20 is an illustration of an illustrative structure that may be utilized to support and position a transducer array of FIG. 19A with respect to a patient.

### DETAILED DESCRIPTION OF THE DRAWINGS

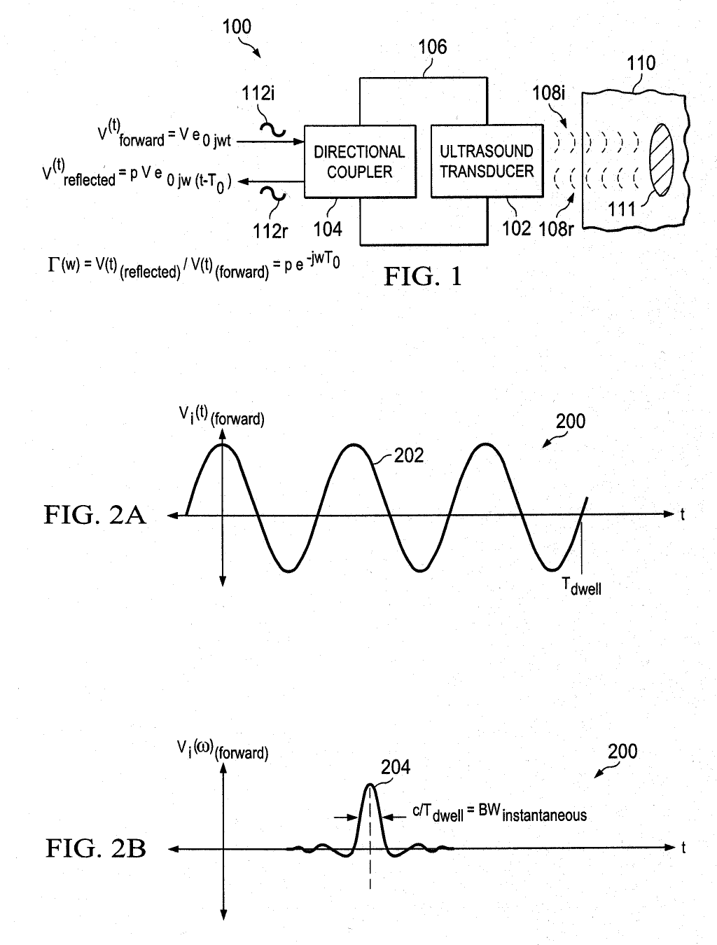

FIG. 1 is a diagram of an illustrative ultrasound probe 100 that utilizes the principles of the present invention. The probe 100 is shown to include an ultrasound transducer 102 and directional coupler 104. A probe housing 106 may be configured to house and/or secure the ultrasound transducer 102 and directional coupler 104, and provide an ultrasound technician or doctor with a structure to hold while performing an ultrasound imaging on an anatomical region 110 of a patient. As an alternative to a single ultrasound transducer 102 with a directional coupler 104, multiple ultrasound transducers (e.g., one for transmitting and one for receiving) may be utilized in accordance with the principles of the present invention. Because the principles of the present invention provide for higher frequency ultrasound injection (e.g., 20 MHz or higher), the use of a transducer for injection of the ultrasound signal and a transducer for receiving reflected ultrasound signals may be utilized because bandwidth for such high frequencies for single transducer to handle both transmission and reception of the ultrasound signals may be too high. It should be understood that the patient may be human or an animal (e.g., dog, cat, cattle, mammal, etc.).

As shown, the ultrasound transducer 102 is configured to communicate an input ultrasound signal (e.g., tones) 108i that are incident signals and receive a reflection signal 108r that is the input signal 108i reflected from a region of interest 111. The tissue features may be cancer cells, micro-calcium structures, tumors, or otherwise, as understood in the art. As further shown, an input voltage signal 112i, such as continuous oscillating voltage signal represented as V(t)forward=V*e₀^{jωt}, may be communicated from a signal generator (not shown) via the directional coupler 104 to the ultrasound transducer 102 to cause the ultrasound transducer to be excited. A reflection voltage signal 112r, such as an oscillating voltage signal represented as V(t)Reflected=V*e₀^{jωt-To}, may be returned from insonified tissue via the ultrasound transducer 102 and directional coupler 104. As understood in the art, a directional coupler is used to separate signals based on the direction of signal propagation. In this case, the directional coupler 104 allows separation of the forward-going wave from the reverse-going wave.

The reflection signal 108 or reverse-going wave is attenuated by a factor, p, which is the fraction of the voltage amplitude that gets reflected back and has a phase shift equal the round trip time delay T₀ that equals the time for the wave to travel to the discontinuity in acoustic impedance and return to the transducer. A reflection coefficient is determined by dividing the reflection signal 108r by the input signal 108i. The divisional of the two terms from the directional coupler 104 allows a voltage amplitude V and time vary components e^{jωt} to cancel. A resulting reflection coefficient is a constant complex number whose magnitude p indicates the fraction of the voltage amplitude that was reflected and the phase of the complex reflection coefficient indicates the round trip time delay. Alternatively, the reflection signal 108r can be multiplied by a signal similar or identical to the input or forward going signal 108i to generate an intermediate frequency (IF) signal (not shown) that may be a baseband signal, as understood in the art. The IF signal or normalized reflection signal may then be integrated for a dwell time T_{dwell} to reduce the noise bandwidth and sampling rate needed. The distance to the tissue feature, represented by a discontinuity of acoustic impedance, may be determined by an equation d=½ CTₒ, where C is the speed of sound in the tissue. The discontinuity in acoustic impedance at distance d can be due to a lesion, anomaly or an anatomic structure that has a different acoustic impedance than the surrounding tissue.

Continuing with FIG. 1, the input signal 112i may be a continuous wave signal V(t)forward=V*e₀^{jωt} being impressed on the ultrasound transducer 102 through the directional coupler 104 at a fixed frequency ωᵢ. The reflected wave or reflection voltage signal 112r is obtained from the directional coupler 104 and ultrasound transducer 102. The reflected waveform is divided by the forward going waveform to obtain the reflection coefficient at the frequency ωᵢ. The reflection coefficient determination process may be repeated for ωᵢ, where i may range from 1 to N, which corresponds to a starting frequency ω_{Min} to a stop frequency of ω_{Max}. The total bandwidth (BW_{Total}) is equal to ω_{Max}−ω_{Min}. Alternatively, the reflection voltage signal 112r may be multiplied by the complex conjugate of the input or forward voltage signal 112i. The complex conjugate of the input voltage signal 112i may be provided to a match filter detector for filtering purposes, as understood in the art. In either case, the resulting signal is integrated for the duration of the transmitted signal, which provides a low instantaneous bandwidth and reduces the noise bandwidth while reducing the sampling rate required.

FIGS. 2A and 2B are graphs of an illustrative continuous wave signal or input signal 200 being impressed on the transducer of FIG. 1 for a dwell time T_{dwell} and corresponding sync function signal that shows a graphical representation of a signal 204 defining instantaneous bandwidth from the input signal 200. The instantaneous bandwidth (BWᵢₙₛₜₐₙₜₐₙₑₒᵤₛ) is inversely proportional to the dwell time. The signal-to-noise ratio (SNR) is proportional to the dwell time T_{dwell}. As understood in the art, the longer the dwell time, the better the resolution of the ultrasound image. In other words, long dwell times increase the SNR and the contrast resolution of each reflection coefficient measured at frequency ωᵢ. The improved SNR allows for deeper penetration, so higher frequencies can be used, and higher frequencies provide greater lateral and axial resolution. As shown, for the computation of the instantaneous bandwidth BWᵢₙₛₜₐₙₜₐₙₑₒᵤₛ, c is a constant that is usually chosen to be unity.

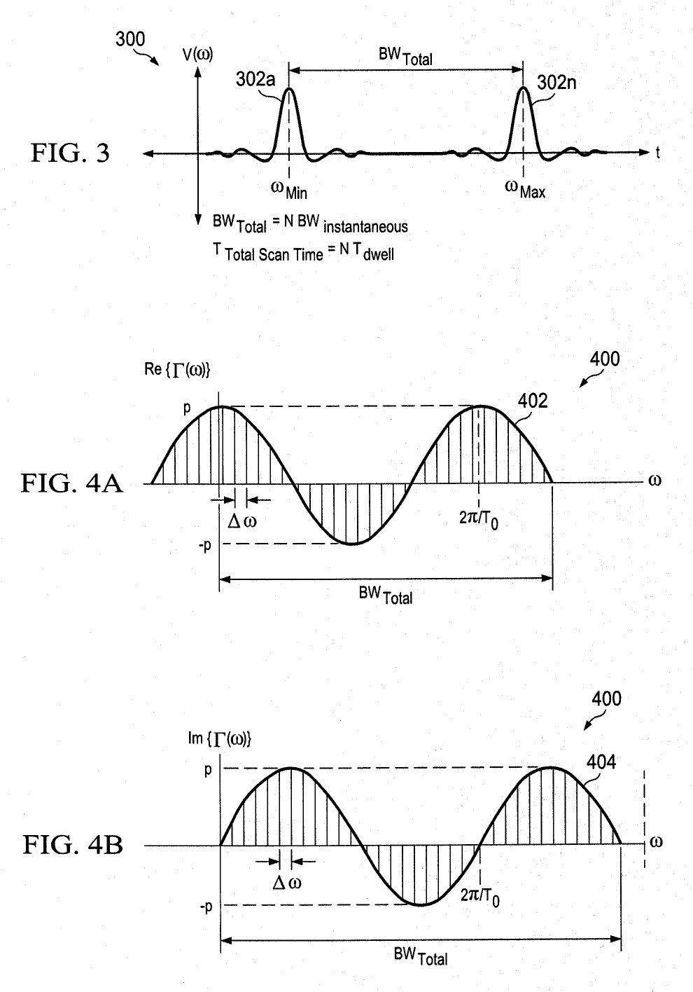

FIG. 3 is a graph showing an illustrative total bandwidth being N times the instantaneous bandwidth based on N frequency values of ωᵢ being stepped from ω_{Min} through ω_{Max}. As shown, two tone signals 302a and 302n centered at frequencies ω_{Min} and ω_{Max} are shown. It should be understood that if N is 100, then 98 additional tone frequencies between ω_{Min} and ω_{Max} may be evenly spaced by (ω_{Max}−ω_{Min})/100. As previously described, larger total bandwidth provides better axial spatial resolution. Narrow bandwidth used for the instantaneous bandwidth allows for the use of inexpensive RF analog electronics and analog to digital converters (ADCs) to be used, thus providing spatial and contrast resolution comparable to much more expensive ultrasound systems for a fraction of the cost. Total scan time may be computed by T_{total scan time}=N*T_{dwell}.

Another feature that results from using a set of continuous tones as input signals is the improvement in acoustic impedance estimates as a function of frequency. The acoustic impedance Z(ω) is obtained from the reflection coefficient array corresponding to each pulse since: Γ(ω)=(Z_{L}(ω)−Z₀)/(Z_{L}(ω)+Z₀), where Γ(ω) is approximately constant over the instantaneous bandwidth, but can vary with w over the total bandwidth. A rational model may be used a parametric model for Γ(ω): Γ(ω)=N(ω)/D(ω), where N(ω) and D(ω) are polynomials in ω. Vector fitting and/or Marquardt Levenberg least squares methods may be used to fit the recorded bandwidth. The rational function may then used to interpolate and extrapolate missing values of Γ(ω). Extrapolated frequency information further increases spatial and contrast resolution.

Continuing, processing gain of a stepped frequency continuous wave (SFCW) ultrasound imaging system stems from the fact that the return signals of duration T_{dwell} are coherently integrated over the total sweep duration T_{Total}=NT_{dwell}, producing an effective noise bandwidth equal to 1/T_{Total} Hz. The principles of the present invention has a typical total sweep period of 1 sec, which yields an effective noise bandwidth of 1 Hz. The typical bandwidth scanned is 50 MHz, which yields a processing gain of approximately 47 dB (i.e., Gp(dB)=10×log(50×10̂6/10̂3)=47 dB).

The processing gain effectively adds about 8 bits (6 dB per bit) of accuracy to the measured signal over the ambient noise. This added accuracy allows for more accurate estimation of salient tissue characterization parameters impedance (Z), velocity of sound in the tissue (V), and attenuation (α), which may be indicative of cancer cells being imaged by an ultrasound system. The longer the integration time the higher the measurement accuracy. However, the total sweep time is limited by how long the transducer can be held in one place on the body without motion of the transducer or the area of the body that is of interest or region of interest.

The stepped frequency continuous wave (SFCW) ultrasound imaging system disclosed herein may be implemented in a number of configurations. One configuration may include a single piezoelectric transducer connected to a directional coupler that allows transmit and receive signals to occur simultaneously. The transducer is excited with a tone, and a matched filter may be used to obtain the in-phase I and quadrature Q components of the returns by integrating over the dwell time T_{dwell}. The I and Q reflection coefficients are high SNR measurements, as described above. These reflection coefficients may be put into a frequency domain vector and an inverse Fourier transform may be used to transform into the time/distance domain. An alternative approach may be to use two transducers one for transmit and the other for receive. The receive signal is used to calculate the I and Q components as before. The time domain waveform is equivalent to a conventional A-scan. The SFCW A-scan improves resolution and accuracy as compared to conventional pulse-type ultrasound imaging systems.

Furthermore, the use of a stepped frequency continuous wave signal generator or Frequency domain reflectometer may be used to measure the reflection coefficient at each time/distance along a path traveled by an incident wave by using a set of frequencies to measure the complex I and Q frequency domain response of the path. The reflection signal composed of the I and Q parameters can be collected and transformed using a inverse FFT to get a time domain signal of the reflection at each point. More energy can be transmitted over time so better SNR and the bandwidth results. In addition, as a result of providing more energy in the ultrasound signal, the transmission can be controlled better in the frequency domain so better resolution results. As previously described, a directional coupler may be utilized to enable using a single transducer capable of transmitting and receiving ultrasound signals.

The harmonics may be measured from a composite return signal as separate reflected signals at 2×, 3×, 4× and 5×, for example, the transmit frequency by using filters in the receiver to measure each harmonic. Each of these I and Q channels are collected in frequency domain and transformed to give a time domain signal at each distance that represents how much second-order non-linearity exists at each time/distance, how much third-order nonlinear exists, and so forth. For every time/distance sample collected, the returns for the transmit frequency, first harmonic, second harmonic, etc. may be collected, and features that are derived from these such as calculations of the impedance Z at every point may be added, the amount of non-linearity may be normalized so b/a and c/a for the first harmonic may be divided by the reflected signal, the second harmonic may be divided by the reflected signal, and so forth. In one embodiment, the velocity of sound at each point may be calculated and used as a tissue characterization parameter. In addition, attenuation at each point may be computed and used as a tissue characterization parameter. These tissue characterization parameters may be used as input to a classifier, such as a neural network, Gaussian mixture model, or combination thereof to output a number or value at each distance that represents the probability of the point in the tissue containing cancerous tissue

FIGS. 4A and 4B are real and imaginary graphs showing illustrative reflected signals or patterns 402 and 404, respectively, that may be observed for measured reflection coefficients over a range of frequencies for a single reflector. The reflection coefficient at each frequency is measured and a complex number measured at each frequency is recorded from the directional coupler, if utilized. The complex number may be stored in an array by a computing system. The array of reflection coefficients may form a complex sinusoidal pattern, such as the one indicated in FIG. 2, where the frequency of the sinusoid indicates the depth of the tumor (i.e., the higher the frequency the further away the target), and the amplitude of the sinusoid indicates the strength of the reflection (i.e., how large the mismatch in acoustic impedance is between the lesion or other reflector and the surrounding tissue).

More specifically, the reflected signals 402 and 404 are discrete sets of reflection coefficients measured at the frequencies from ω_{Min} to ω_{Max}, and include a set of complex sinusoids. The amplitudes of the reflected signals 402 and 404 correspond to the reflection coefficients and the frequencies correspond to distances. The reflected signals 402 and 404 may be observed by using an inverse Fast Fourier Transform (FFT) of the set of the reflection coefficients, as shown.

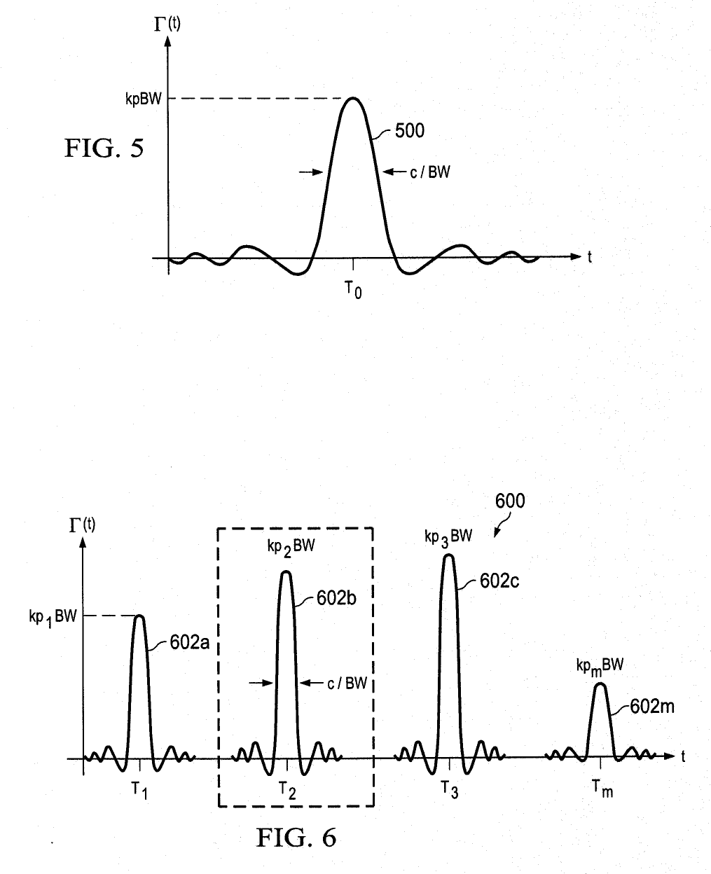

FIG. 5 is a graph showing an illustrative sinc function signal 500 in the time domain that defines a tone signal and that may be utilized to define a window for measuring a region of interest. The sinc function signal 500, mathematically described by sin(x)/x, results from taking the Inverse Fast Fourier transform (IFFT) of the reflection coefficient array resulting from an ultrasound scan that yields a pulse (sync function signal 500) at the distance that the lesion is from the transducer. The pulse amplitude corresponds to the strength of the reflection, in this case the amplitude of the pulse is proportional to the bandwidth BW. That is, each pulse observed in the inverse FFT has a height that is proportional to the total bandwidth in the reflection coefficient and a width that is inversely proportional to the total bandwidth. Thus, the larger the total bandwidth the narrower the pulse and the better the spatial resolution. Where k in FIG. 5 is a constant. The sync function signal 500 is equivalent to a conventional A-scan performed by conventional pulse ultrasound systems. With conventional ultrasound scans, a pulse of RF ultrasound is transmitted into the tissue. Discontinuities in acoustic impedance reflect the pulse back to the transducer. The principles of the present invention are different from the conventional ultrasound in that, in this case, continuous tones are transmitted into the tissue and complex reflection coefficients are recorded as a function of frequency. The frequency measurement approach allows more energy to be put into the tissue, so deeper penetration into the tissue is possible. The attenuation of acoustic energy with higher operating frequency can thus be mitigated by increasing the energy used over a longer scan time. The longer scan time allows regulated limits to be imposed on peak energy used.

The attenuation in ultrasound imaging is usually between 0.5 dB/cm MHz and 1 dB/cm MHz. So, for a system with a maximum frequency of 10 MHz, every additional centimeter of penetration requires another 5 to 10 dB increase in SNR. The principles of the present invention can easily increase the SNR by 10 dB to 100 dB, which provides several centimeters of extended measurement range to accurately locate and analyze ROIs (e.g., cancer or other pathologic cells). The extended range is generally best traded for a higher operating frequency since the higher frequency gives better spatial and contrast resolution. The principles of the present invention allow the operator to obtain the desired tradeoff via software and/or hardware control.

FIG. 6 is a graph that shows multiple reflection signals 600 in the time domain from using an ultrasound system with continuous tones in accordance with the principles of the present invention. As a result of having multiple reflection signals 600, each region of interest can be separated and analyzed separately. The reflection signals 600, which are sync function signals, may be distinctly isolated as signals 602a-602m being centered at times T₁ . . . Tₘ, respectively. The discrete times T₁ . . . Tₘ provide the distances of the potential regions of interest, and the amplitudes represent the reflection coefficients of the respective regions of interest (i.e., the reflectors). This waveform is the equivalent of an A-scan in ultrasound. This high resolution, high signal-to-noise ratio version of an A-scan can be used to create a high fidelity B-scan or three-dimensional views or many other visualization modalities that are generally derived from A-Scans.

Acoustic impedance estimates as a function of frequency provide increased resolution over conventional ultrasound systems. The acoustic impedance Z(ω) may be obtained from the reflection coefficient array corresponding to each pulse since:

Γ(ω)=(Z_{L}(ω)−Z₀)/(Z_{L}(ω)+Z₀), where Γ(ω) is approximately constant over the instantaneous bandwidth, but can vary with w over the total bandwidth. A rational model is used for Γ(ω), as Γ(ω)=N(ω)/D(ω), where N(ω) and D(ω) are polynomials in ω. Again, vector fitting and/or Marquardt Levenberg least squares methods may be used to fit the recorded bandwidth. The rational function is then used to interpolate and extrapolate missing values of Γ(ω). The extrapolated frequency information further increases spatial and contrast resolution.

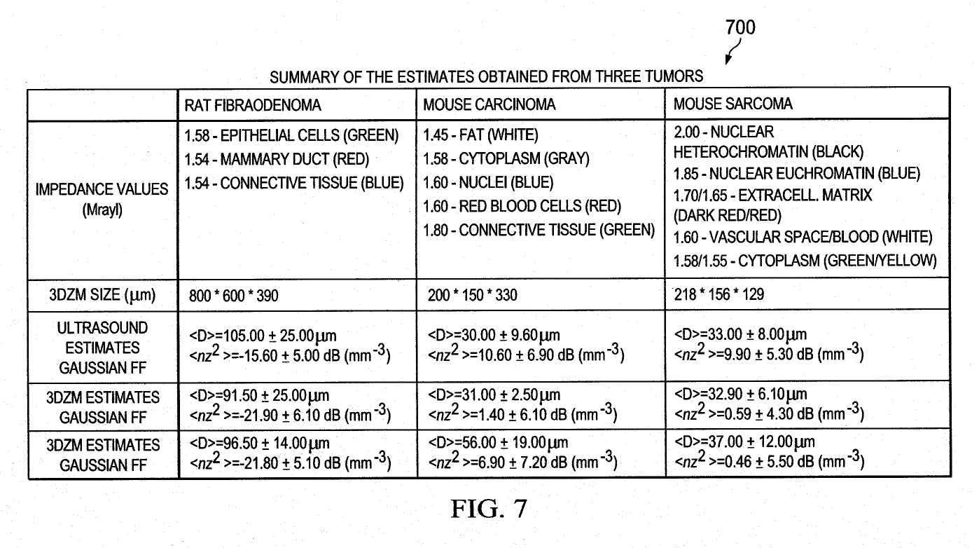

FIG. 7 is a chart 700 that shows the typical variation in acoustic impedance found between cancerous tissues and benign tissues. This chart 700 shows the difference between cancer and non-cancer tissues as seen from the acoustic impedance in MRAYL.

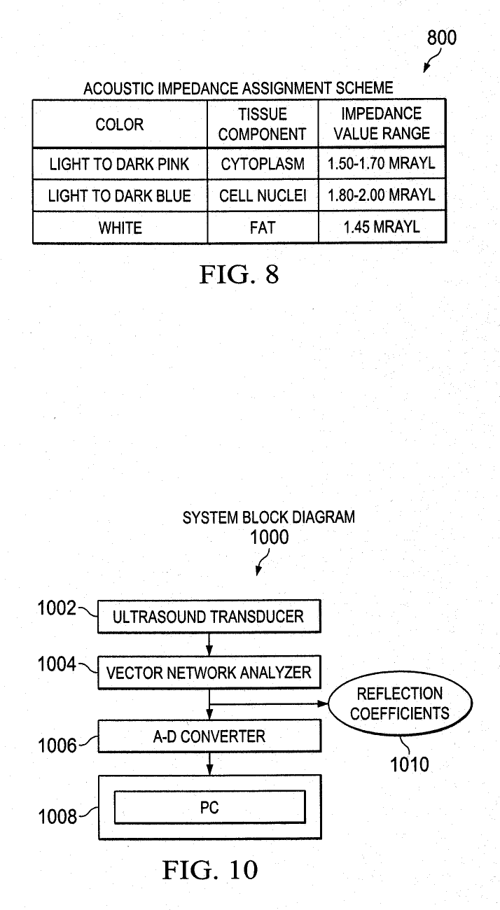

FIG. 8 is a chart 800 that illustrates a typical color code used to help visualize the acoustic impedance so an ultrasound technician can better identify cancerous tissue from an ultrasound image. The data in charts 700 and 800 demonstrate that cancer tissue has a different acoustic impedance than the surrounding tissue, which, when combined with shape, may be used by the operator to help determine when cancer or other pathology might be present.

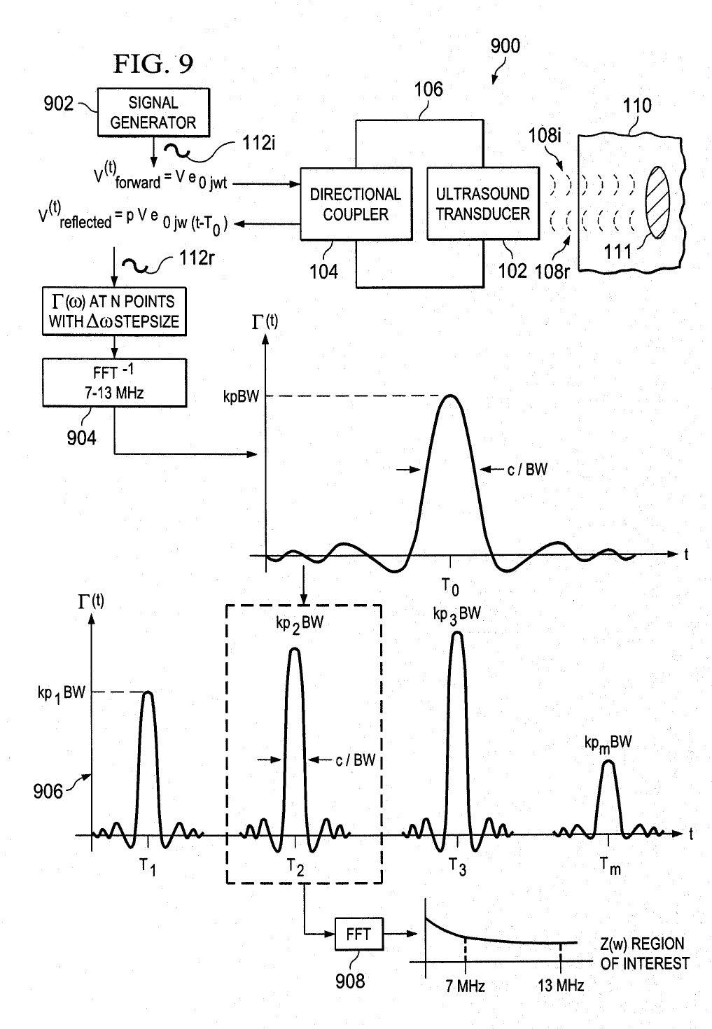

FIG. 9 is a block diagram of an illustrative ultrasound system 900 that includes a signal generator 902, which may generate a continuous wave sinusoid 904 that is communicated through the directional coupler 104 to the ultrasound transducer 102. Alternatively, a two element transducer where one is used to transmit and the other used to receive could be used. The two element can be collocated in one housing or located separately in a configuration where the tone transmitted by one element is received by the other element. The frequencies used may be stepped from a minimum to maximum frequency by stepping through a number of N steps, where each frequency is dwelled on for a time T_{dwell}. At each individual frequency, a complex reflection coefficient may be measured and stored. The ultrasound scanning process may be continued for N reflection coefficients. An inverse FFT 904 is obtained to identify the number of reflectors, including distances and amplitudes. The individual pulse corresponding to each region of interest (i.e., reflector) may then be shifted to the origin in the time/space domain for analysis and windowing at 906 using a processing unit (e.g., processor executing software), and then converted back to the frequency domain using an FFT 908. The inverse FFT 904 and FFT 908 may be hardware or hardware executing software, as understood in the art.

Each individual lesion may then represented with a rational model and an acoustic impedance spectrum may be interpolated and extrapolated from the model. The extrapolated acoustic impedance may be used by a computer aided diagnostic (CAD) software system to aid the technician in determining cancer versus benign tissue, for example. The extrapolated impedance may also used to reconstruct a high-resolution synthetic A-Scan, which provides the technician with improved spatial and contrast resolution to help visualize the tissues. The improved resolution imaging aids the clinician in finding micro-calcifications in breast tissue, for example, which indicates early stages of breast cancer. In other words, the improved contrast helps identify lesions that may be cancerous.

FIG. 10 is a block diagram of an illustrative ultrasound system 1000 that includes an ultrasound transducer 1002, vector network analyzer (VNA) 1004 or similar analog front-end, analog-to-digital (A-D) converter 1006, and computing device 1008. The vector network analyzer 1004 may be configured to generate reflection coefficients 1010, as previously described, that are a series of complex coefficients responsive to regions of interest being scanned or imaged. The computing device 1008 may be a personal computer, mobile device (e.g., mobile phone that operates an app configured to read and process the reflection coefficients 1010), tablet, or any other electronic device configured to process and/or display ultrasound images scanned by the ultrasound transducer 1002. Because the ultrasound transducer 1002 may be inexpensive and the computing device 1008 may be relatively inexpensive (e.g., mobile phone, tablet, etc.), the overall cost of the ultrasound system 1000 may be inexpensive enough for low-income regions of the world to purchase or otherwise obtain.

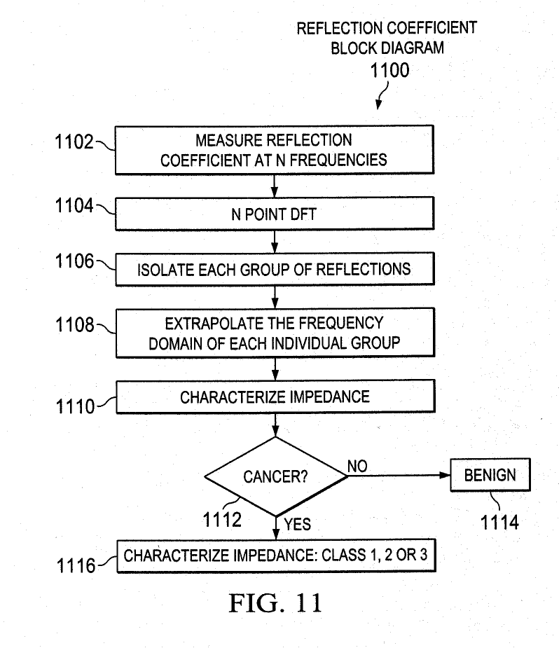

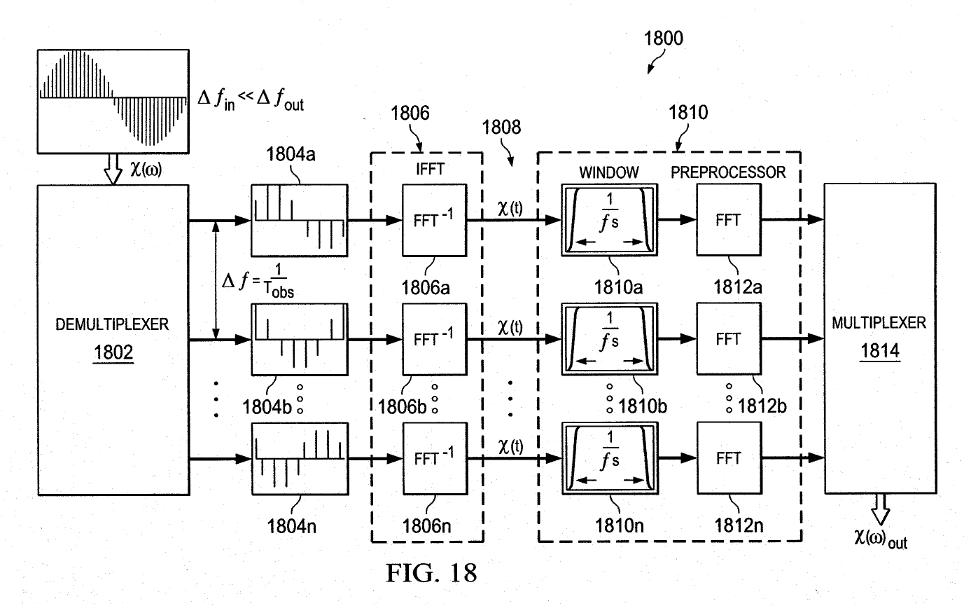

FIG. 11 is a flow diagram of an illustrative diagnostic process 1100 that may be utilized by the ultrasound system of FIG. 10 in performing imaging and analysis for determining whether cancer, for example, exists in imaged tissue of a patient. The diagnostic process 1100 is a portion of an overall diagnostic process described with respect to FIG. 18. The diagnostic process 1100 may start at step 1102, where reflection coefficients are measured at N frequencies in the frequency domain. The reflection coefficients may be stored in a reflection coefficient array. At step 1104, an N-point inverse discrete Fourier transform (DFT) may be performed to convert reflection coefficients from the frequency domain into the time domain. In one embodiment, each group of reflection coefficients in the time domain (i.e., representing reflections from different regions of interest) may be isolated at step 1106. The time domain signal can be windowed, thereby isolating regions of interest based on the general appearance of the signal locally. The windowed region of interest may then be transformed into the frequency domain by using a discrete Fourier Transform, as understood in the art. At step 1108, the frequency domain of each individual group of reflection coefficients may be extrapolated. At step 1110, each component of the acoustic impedance may be characterized or modeled as a rational function, thereby providing for extrapolation and interpolation of the acoustic impedance (or other characteristic) of each lesion using the rational model. An ultrasound image may be visualized and automatic pattern recognition methods may be utilized to help the technician identify regions of interest in the ultrasound image that may be indicative of cancerous tissues.

At step 1112, a determination may be made as to whether the impedance characteristics (or any other characteristics representative of the imaged region of interest) are indicative of cancer being present. If the determination at step 1112 is negative, then the process continues at step 1114, where the region of interest is determined to be benign. If the determination at step 1114 is positive, then the process continues at step 1116, where the impedance may be characterized as being of a certain class, such as class 1, 2, or 3. In one embodiment, a neural network or other learning or identification algorithm may be utilized in accordance with the principles of the present invention.

The acoustic impedance may be interpolated down to low frequencies. Acoustic impedance at low frequencies is generally indicative of the elasticity of the mass (e.g., tumor). As understood in the art, cancer tissue is general much stiffer than benign tissue and so the acoustic impedance, particularly the imaginary part of the acoustic impedance, can be used as an indication of cancerous tissue in a pattern recognition system. Through the principles of the present invention, the operator training and skill level required may be modest compared to conventional ultrasound systems due to the ability to provide recommendations based on automatic pattern recognition methods to help the technician through the analysis of the ultrasound data.

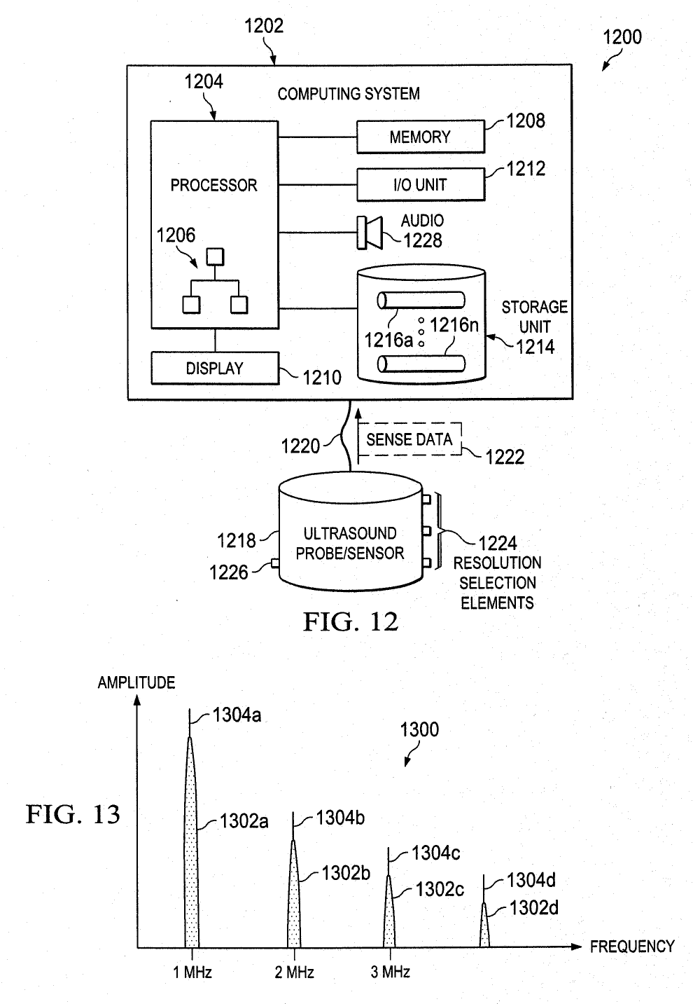

FIG. 12 is an illustration of an illustrative ultrasound system 1200 that may utilize the principles of the present invention to image and detect cancer in tissue of a patient. The ultrasound system 1200 may include a computing system 1202 that includes a processing unit 1204 configured to execute software 1206. The computing system 1202 may be a specialized piece of equipment configured for the sole purpose of performing ultrasound processing. Alternatively, the computing system 1202 may be a general piece of hardware that operates an application or app, if on a mobile device, that is configured to perform functionality (e.g., display ultrasound images, data, allow for window selection, etc.) of an ultrasound system. The processing unit 1204 may include one or more processors, and the software may be configured to perform processing of ultrasound image data collected by the system 1200 or simply display processed image data. The processing unit 1204 may be in communication with a memory 1208 configured to store data and software, display configured to display ultrasound image data, input/output (I/O) unit 1212 configured to communicate ultrasound image data locally or via a network, and storage unit 1214. The storage unit 1214 may be configured to store data repositories 1216a-1216n (collectively 1216), which may store ultrasound image data collected from patients.

An ultrasound probe or sensor 1218, such as one configured as shown in FIG. 1, may be in communication with the computing system 1202. In one embodiment, the ultrasound probe 1218 may be connected to the computing system 1202 via a cord 1220, which may be permanent or removable. Alternatively, the ultrasound probe 1218 may communicate wirelessly using any wireless communications protocol (e.g., 802.11, Bluetooth, etc.) as understood in the art. Sense data 1222 sensed by the probe 1218 may be communicated to the computing system 1202 for processing by the processing unit 1204. Alternatively, probe 1218 may be configured to process ultrasound image data and the sense data 1222 may be processed image data for communication and display on the display 1210 of the computing system 1202.

The probe 1218 may further include one or more resolution selection elements 1224 that enables a medical technician to select resolution of the probe 1218. The resolution selection elements 1224 may cause the probe 1218 to use longer dwell time, use additional frequency steps, have higher bandwidth, or otherwise. The probe 1218 may include one or more sensory device 1226 that may be used to notify the user of a variety of different functions. The sensory device(s) 1226 may include an illumination device (e.g., light emitting diode), audio device (e.g., speaker), or motion device (e.g., vibrator). The functions may include start scan, scan complete, move probe, cancer detected, no cancer detected, unknown mass detected, battery status (if battery powered), or otherwise. In an alternative embodiment, an audio device 1228, such as a speaker, may be utilized to provide the technician with audio, such as tones, synthetic voice, or otherwise, to provide ultrasound operation and diagnostic information (e.g., “no cancer detected,” “scan complete,” “processing scan,” etc.).

FIG. 13 is a graph showing an illustrative RF transmission line spectrum 1300 responsive to a non-linear tissue when excited by an ultrasound tone or signal. As shown, a fundamental tone 1302a centered at a center frequency 1304a, in this case 1 MHz, is shown to be reflected from a nonlinear lesion that results in harmonics 1302b-1302d centered at center frequencies 1304b-1304d. The center frequencies 1304b-1304d are positioned at multiples of the center frequency 1304a of the fundamental tone 1302a (i.e., 2 MHz, 3 MHz, and 4 MHz, respectively). The harmonics 1302b-1304d may include more than three harmonics, and may be used to provide additional characteristic parameters of the nonlinear lesion beyond attenuation that is provided by the fundamental tone 1302a.

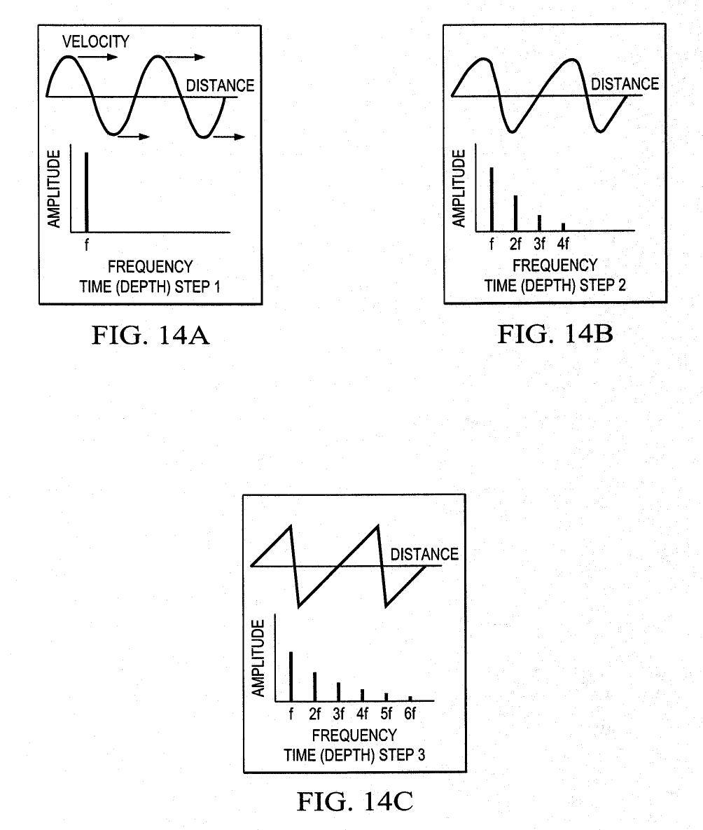

FIGS. 14A-14C are graphs showing a time sequence that illustrates the generation of harmonic frequencies. Time step 1 shown in FIG. 14A contains a wave of single frequency. As the wave travels into the tissue at time step 2 shown in FIG. 14B, the wave becomes distorted. Additional frequency components are created that are integer multiples of the initial frequency. These components are called harmonic frequencies. As the wave continues to travel, the wave becomes highly distorted at time step 3 shown in FIG. 14C, and further becomes very rich in harmonic frequencies. Note that the harmonic frequencies are created and accumulate as the wave travels through the tissue. Although many harmonic frequencies are produced with nonlinear wave propagation, the amplitudes of the higher harmonics are extremely small. Therefore, the principles of the present invention use the second harmonic (2f), which is twice the nominal transmitted frequency.

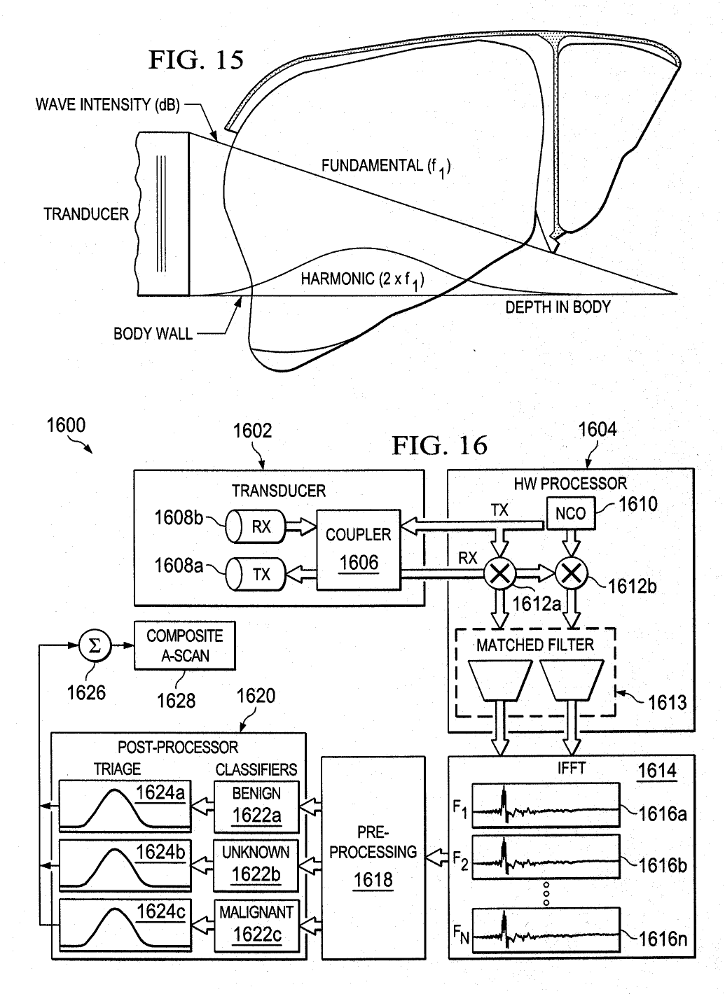

FIG. 15 is a schematic that depicts relative intensities and frequency changes of harmonic ultrasound beams and fundamental transmitted waves with increasing depth in tissues. A frequency stepped continuous wave versus return signal includes a round trip reflection from each interface with a change in acoustic impedance that occurs at the transmit frequency. In addition, there is a first harmonic at twice the fundamental transmit frequency, a third harmonic at three times the fundamental transmit frequency, and so forth. The principles of the present invention measure not only the return or reflected signal at the transmit frequency, but also the return or reflected signal one or more harmonic frequencies. The amount of each harmonic may be calculated using an inverse FFT to calculate the amount of each harmonic at each axial position along the scan. The fundamental, first harmonic, second harmonic, and third harmonic at each distance (sample by sample) may be used to classify the probability of cancer, benign tissue, or other type of tissue or substance is present at that time/space sample. A substance may be any tissue or matter that is within an object being ultrasound scanned, and is generally meant to mean that the substance provides a reflection of the ultrasound signals, thereby being able to be identified with some probability by the ultrasound system in accordance with the principles of the present invention.

The reflected pulses measured as the ultrasound input pulse travels though the various types of tissues are distorted in that each frequency component of the pulse travel at a different speed (dispersion) and each frequency component is attenuated by a different amount due to the frequency dependent attenuation factor exp((−a₁ω+a₀)x) and the reflected pulses are filtered by the superposition of reflected pulses from nearby anatomic structures. Thus, the calculation of frequency domain quantities by transforming the reflected pulses of conventional echo pulse ultrasound imaging systems by the FFT, for example, yields unreliable estimates. Quantities found to give tissue characterization information are generally frequency domain quantities.

Four tissue characterization or characteristic parameters that may be used in determining tissue type from ultrasound scans may include one or more of the following parameters:

1) attenuation coefficient a₁ from the attenuation factor, which is exp((−a₁ω+a₀) and may be measured by taking the slope of ln(X(ω));

2) Harmonic content, which is the response at 2ω for each transmitted frequency;

3) Acoustic impedance—the reflection coefficient as a function of frequency can be used to determine the acoustic impedance as a function of frequency, which is cumulative integrated to get the absolute acoustic impedance at each point; and

4) Speed of sound in each segment of tissue.

With conventional pulse ultrasound systems, as a result of using pulses, the pulse-based estimates in the frequency domain are noisy and include distortion. The principles of the present invention utilized a stepped frequency continuous wave input signal and perform measurements in the frequency domain, so signal-to-noise ratio is higher, resolution is higher, and penetration into tissue is deeper.

The ultrasonic attenuation coefficient is a parameter that may be used to characterize tissue pathologies. The spectral difference method, the spectral log difference method, and the hybrid method, as understood in the art, may be used for estimating the attenuation in human tissue, or mediums of other objects (e.g., animal tissue, land masses, infrastructure, such as pipes, etc.). The spectral difference method uses the decrease of the different frequency components of the power spectrum with respect to depth to estimate the attenuation coefficient. The spectral log difference method finds the attenuation by calculating the slope of a straight line that fits the log ratio (difference between log spectra) of the two power spectra from the proximal and the distal segments of the region of interest (ROI). The hybrid method estimates the attenuation coefficient slope by measuring the downshift in the center frequency of the spectra with depth after multiplying by a Gaussian filter. The accuracy and the precision of these methods are strongly dependent on the ROI size (the number of independent echoes laterally and the number of pulse lengths axially) and on the level of homogeneity within the ROI. These methods use 10 to 20 pulse lengths per ROI in order to provide reasonable accuracy of the attenuation coefficient. This condition is easy to meet in an in vitro environment, where the tissue is extracted and put in a test chamber. However, it is very difficult to obtain in vivo.

The Fourier transform of the windowed waveform is given H(d, ω), where d is the distance to the window position in the body and the speed of sound S is given by S(ω)=ωd/[arg{H(d, ω)}] and the attenuation is given by α(ω)=−log|H(d, ω)|/d. The speed of sound and attenuation may then transformed into the time/space domain using the inverse Fourier transform. The resulting time/space domain waveforms for the speed of sound S(x) and attenuation coefficient α(x) may be displayed in similar manner to A-scans, which indicate tissue characterization through the tissue along the scan path. Parametric and non-parametric methods may be used in the calculation involving H(d, ω) to obtain the attenuation and speed of sound tissue characterization parameters. The high fidelity calculations of local tissue attenuation and speed of sound can be used to correct or compensate the A-scan before display so as to enhance the fine details found to be the most indicative of pathology in tissues, as further described below.

The acoustic impedance can also be calculated from H(d, ω), where Zi(ω)=(1−H(d,w))/(1+H(d,w)) Zi(w), where Zi is the acoustic impedance of the previous surface. The acoustic impedance of initial surface Zo(ω) is known and is given by the index matching material used on the surface of the skin during the scan. The acoustic impedance Z of each consecutive surface through the body is calculated using equation above for Zi(ω).

In compensating an A-scan based on one or more of the characteristic parameters, the following may be utilized. The A-scan waveform h(t) includes the reflected voltage waveform as a function of time Y(t) sensed by the transducer divided by the transmitted voltage time waveform X(t) going into the transducer. Thus, H(t)=Y(t)/X(t) (eqn. 1). The speed of sound c is usually approximated by a constant, c=1540 M/sec. And, the A-scan is usually displayed as a function of distance along the scan line through the tissue, where H(d)=H(ct). In compensating the A-scan for attenuation effects, by rescaling the time axis in eqn. 1 by the constant c, the attenuation changes the A-scan by decreasing amplitude as a function of frequency and depth. Thus, H(d) measured=A(ω,d)*H(d) actual. Since the attenuation A(ω,d) is calculated as described previously, compensation may be performed by dividing the attenuation factor out of the measured signal to compute the actual signal H(d) actual=1/A(ω,d) H(d) measured. The compensated A-scan can be displayed and provide enhanced emphasis on one of the quantities found to indicate the presence of cancer, namely the attenuation.

In compensating for speed of sound changes in the tissue, c is actually a function of distance d and the more accurate representation is given by H(d)=H(c(d)t), where C(d) is obtained from the inverse Fourier transform of the speed of sound calculation given by −wd/arg(H(ω,d)) as stated previously. The compensation for the change in the speed of sound helps to show subtle deviations in the A-scan that allows the diagnosis of pathologic tissues. Note, both the speed of sound and attenuation compensation methods can be used jointly to increase the subtle details used to detect pathologies, such as cancer, in tissue.

The harmonic measurement may be facilitated by the increase frequency resolution and may be measured as the I and Q components from the demodulation of twice the transmitted frequency. The harmonic content b may be displayed for the region of interest and is generally normalized by the fundamental signal received a. Thus, the non-linearity of the tissue is given by the ratio b/a.

The principles of the present invention allow the ROI to be adaptively obtained in the time domain and then windowed out of the total time trace and transformed into the frequency domain via the FFT. The tilt of spectrum of the ROI may then used to calculate the attenuation coefficient with high accuracy. This highly accurate in vivo calculation has heretofore not been possible by conventional ultrasound systems. The accuracy is further improved by use of the higher SNR of the stepped frequency continuous wave. This technique of using FSCW also allows for pre-planned frequency sounding of the ROI to help increase the SNR in the bandwidth of interest for a high precision examination of the ROI.

FIG. 16 is an illustration of an illustrative ultrasound system 1600 showing processing of reflected ultrasound signals to determine whether cancer exists in tissue being imaged by the ultrasound system 1600. The ultrasound system 1600 includes a transducer 1602 in communication with a processing unit 1604. The transducer 1602 includes a coupler 1606 that communicates with one or more ultrasound transducers 1608a and 1608b for transmitting and receiving ultrasound signals, respectively. As previously described, the transducers 1608a and 1608b may be a single transducer or be individual transducers. The processing unit 1604 may include a numerically controlled oscillator (NCO) 1610 that creates oscillation signals (not shown), such as the ultrasound input voltage signal 112i of FIG. 1. Two mixers 1612a and 1612b, which may be part of a matched filter 1613, may be utilized to produce real (I) and imaginary (Q) parts of reflection coefficients as received from the receive transducer 1608b via the coupler 1606. As understood in the art, the matched filter 1613 may include integrator(s) for use in filtering the down-converted signals. Alternatively, the matched filter 1613 may be performed on non-down-converted signals (i.e., performed on the ultrasound signals as opposed to the IF signals)

The down-converted frequencies may include the transmitted frequency that provides the complex Fourier transform component (Iₒ+QₒJ) of the scan profile at the transmitted frequency. The down-converted frequencies may also include the twice the transmitted frequency that provides the complex Fourier transform component (I₁+Q₁J) of the scan profile at twice the transmitted frequency (i.e., the first harmonic). The down-converted frequencies may also incorporate frequencies at three times, four times, and higher multiples of the transmitted frequency providing the second, third, and higher harmonic responses of the tissues in the scan path. The inverse Fourier transform may each set of scanned data that is (i) the set of N complex numbers I+QJ taken at the transmitted frequency for each frequency step from Fₘᵢₙ to Fₘₐₓ of the scan, (ii) the set taken at twice the transmitted frequency at each of the N frequencies taken from Fₘᵢₙ to Fₘₐₓ, (iii) the set of N complex numbers taken at three times the transmitted frequency at each of N frequencies taken from Fₘᵢₙ to Fₘₐₓ, and (iv) optionally higher harmonic frequencies. The inverse Fourier transform operation converts the harmonic data into the time/space domain, which allows the harmonic information to be visualized at each spatial location along the scan path. The separate time/space waveforms may be combined to provide one composite signal that provides indication of the pathology of the local tissue being scanned.

From the mixers, the reflection coefficients are communicated to a bank of inverse FFTs or IFFTs 1614. Because the ultrasound system performs frequency steps, reflection coefficients at each frequency step are loaded into the bank of inverse FFTs 1614, thereby providing sufficient information for the bank of inverse FFT 1614 to generate time domain signals 1616a-1616n (collectively 1616) at each of the harmonic frequencies. A preprocessor 1618, which is further described with respect to FIG. 18, may receive each of the time domain signals 1616 to identify regions of interest and calculate tissue characteristic parameters, as further described below. The preprocessor 1618 may communicate the tissue characteristic parameters to a post-processor 1620 that may include classifiers 1622a-1622c (collectively 1622) that may be used to automatically determine probabilities of what the regions of interest actually are (e.g., malignant cancer, benign cancer, unknown tissue). The classifiers 1622 may include neural network(s), as understood in the art, that may be trained and learn over time to assess the regions of interest. Triage modules 1624a-1624c (1624) may receive data from the classifiers 1622 for aggregation by a summer 1626. Although classifiers 1622 and triage modules 1624 are shown, it should be understood that alternative and/or additional modeling processes may be utilized to automatically assist in assessing the regions of interest in accordance with the principles of the present invention. The resulting aggregation or summation may be displayed as a composite A-scan 1628. It should be understood that other types of outputs, such as B-scans (not shown), Boolean outputs (e.g., cancer or no cancer) via an indicator (e.g., LED and/or audio), and otherwise, may be generated by preprocessor 1618 and post-processor 1620. As an example, and as further described, A-scans with compensation based on tissue characterization parameter(s) may be displayed for a user or operation of the ultrasound system.

To build the set of classifiers 1622 based on this system, the following steps may be carried out:

1) Frequencies 1 MHz-15 MHz are launched into the body (human, animal, or otherwise). It should be understood that the principles of the present invention may support higher frequencies.

The matched filter 1613 may be used both on the fundamental tone that is injected, as well as the harmonics of the injected tone received by the transducer 1608b. It should be understood that a separate matched filter may be used for each individual frequency.

2) The output of the matched filter yields the I and Q signal (i.e., set of reflection coefficients) for not only the fundamental frequency, but also the harmonic frequencies that are scanned and processed by the matched filter. This yields two matrices, one for the I and another for the Q signals that are: {harmonic scanned}×{number of scanned frequencies}. For example, if 10 harmonic frequencies are scanned and 1000 fundamental frequencies are used, the resulting measurement matrix is 10 rows by 1000 columns. This matrix can then be used for further processing.

3) The IFFT 1614 then uses each row in a similar fashion. The result is a set of A-scans for the fundamental frequency and may include one or more harmonics that can be viewed individually or in combination to provide an indication as to the pathology of the underlying tissues.

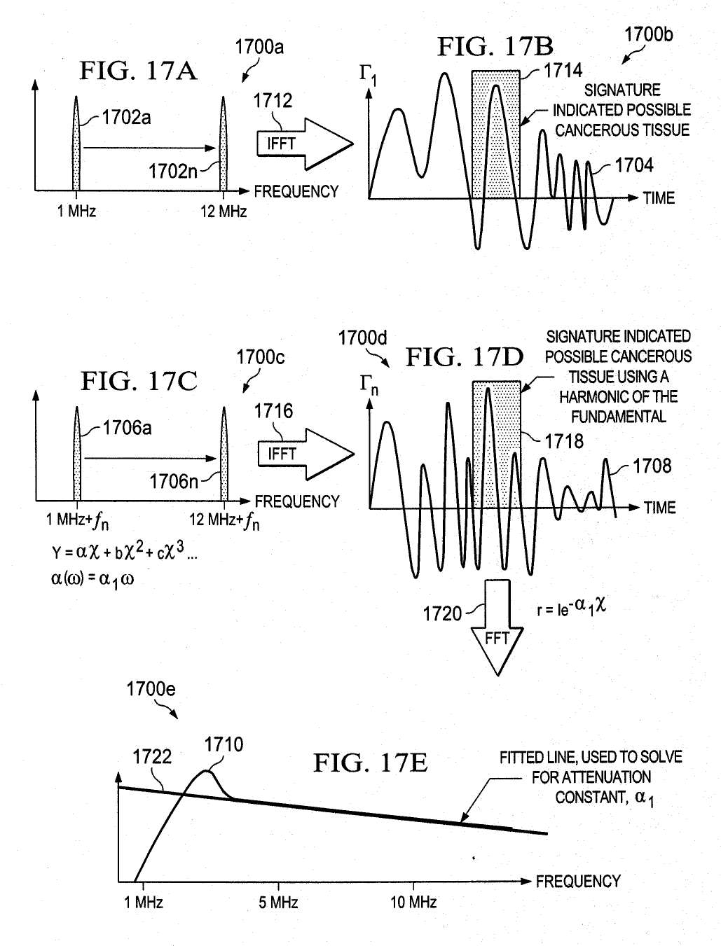

FIGS. 17A-17E are graphs 1700a-1700d of illustrative signals 1702, 1704, 1706, 1708, and 1710 that are used in performing an ultrasound scan and determining a tissue characteristic, in this case an attenuation constant α₁, to determine the type of tissue being scanned (e.g., malignant cancer). FIG. 17A shows a graph 1700a that includes a set of tones 1702a-1702n (collectively 1702) that are stepped from a low frequency (e.g., 1 MHz) to a higher frequency (e.g., 12 MHz). The set of tones 1702 that are injected into a patient may be reflected from a region of interest (e.g., tissue discontinuity) that is processed by an IFFT 1712 to produce a reflection signal 1704 in the time domain, as shown in FIG. 17B. The ultrasound system may automatically or be manually controlled to identify a signature that indicates possible cancerous tissue. In identifying the signature, a window 1714 may be used to isolate the signature portion, thereby enabling the system to better determine location and size of the cancerous tissue. FIG. 17C shows a set of tones 1706a-1706n that are harmonic tones from the set of tones 1702 reflected from the region of interest. An IFFT 1716 may be used to convert the harmonic tones 1706 into the signals 1708. As shown in FIG. 17D, similar to the window 1714 of FIG. 17B, a window 1718 may be used to isolate or focus on a signature that indicates possible cancerous tissue.

From the time domain signals 1704 and 1708, an FFT 1720 may be used to generate a frequency signal 1710 that is representative of attenuation in the frequency domain. A fitting algorithm, such as a polynomial fit, may be used in determining the attenuation constant α₁ of the tissue in the ROI.

FIG. 18 is a block diagram of an illustrative portion of an ultrasound system that includes the preprocessor of FIG. 16. As shown, a reflected frequency signal x(ω) from a stepped frequency input signal is received from a transducer of the ultrasound system. A demultiplexer 1802 demultiplexes the signal into the component frequency samples 1804a-1804n (collectively 1804) that are separated in the frequency domain by Δf or 1/T_{obs}. The demultiplexer separates or subsamples (e.g., selects every third frequency) the stepped frequency components from one another, and any other technique for separating the frequency components, such as deinterleaving, may be utilized in accordance with the principles of the present invention. It is presumed that the stepped frequency spacing is less than the frequency resolution required by the time duration of the ROI so Δfₛ<<Δfₒᵤₜ, where Δfₛ is the stepped frequency spacing and Δfₒᵤₜ is the reciprocal of the time duration of the ROI with respect to the demultiplexer 1802. A set of inverse FFTs 1806a-1806n (collectively 1806), one for each the fundamental and each harmonic frequency, may be used to generate respective time domain signals x(t) 1808.

A preprocessor 1810 that includes windows 1810a-1810n (collectively 1810) may be utilized to focus on a signature portion of the samples 1804 in the time domain. In one embodiment, the windows 1810 may be created automatically (e.g., selecting the 3 dB points on either side of a peak in the time domain signal (see, for example, FIG. 17)). Alternatively, the windows 1810 may be selected manually by a user creating a window on an image to focus on a particular region of interest. By performing the windowing function in the time domain based on high resolution, high SNR measurements in the frequency domain, a high-degree of accuracy may be made in determining tissue characteristics within a specified ROI. The signal data selected within the windows 1810 may be converted back to the frequency domain by FFTs 1812a-1812n (collectively 1812). From the FFTs 1812, a multiplexer 1814 may aggregate the individual time domain signals into a windowed frequency signal x(ω)ₒᵤₜ for post-processing. As with the demultiplexer 1802, but in an opposite manner, the multiplexer 1814 is representative of a function that combines the demultiplexed signals or frequencies and interleaves or otherwise combines the signals.

In summary, FIG. 18 shows how the principles of the present invention provide for high frequency resolution (highly spaced independent frequency samples in the frequency domain) of a scan with a high spatial/temporal resolution over a small region of interest, such as a tumor. By way of contrast, conventional pulse echo systems cannot do provide for high spatial/temporal resolution over a small region of interest since conventional pulse echo systems have a limit on the frequency resolution due to the finite duration of the pulse, which is on the order of 1 MHz. In accordance with the principles of the present invention, the use of a stepped frequency continuous wave (SFCW) ultrasound that integrate tones for longer periods of time (e.g., 1 ms) substantially eliminates the constraint on frequency resolution as are present with conventional ultrasound systems, as described above.

Furthermore, FIG. 18 shows how deconstruction and reconstruction of the ultrasound signals from and to the frequency domain such that an arbitrary small region of tissue can be examined with the same high frequency resolution that was used in the frequency scan that is KHz resolution. This high frequency resolution and high SNR due to large integration time of each tone provides high accuracy calculation of the signals used for tissue characterization.

As a more detailed example, a piezo transducer may scan from 5 MHz to 15 MHz in 10 KHz increments to give (15-5)×10̂6/10×10̂3=1000 scans. Each scan may last approximately 1 msec, so the total time for 1 scan is 1 second. If a 1.5 mm length of tissue is windowed out of the full scan for high frequency resolution analysis then a time window of Tωᵢₙ=X/c=1.5×10̂−3 M/1500 M/sec=1 μsec=1×10̂−6 seconds is taken. The frequency samples may therefore be spaced at Δf=1/Tωᵢₙ=1 MHz to satisfy the Nyquist criteria. Thus, to cover the bandwidth from 5 to 15 MHz uses 10×10̂6 samples/10̂6 Hz=10 samples per band. As a result, and, by way of non-limiting example, the 1000 frequency samples may be subsampled into 100 sub-scans of 10 samples each, where each coarse frequency scan is staggered by 10 KHz from the next sub-scan. These 100 sub-scanned sequences may be windowed in the time domain to extract the approximate 1.5 mm region of interest and convert that window of data back into the frequency domain. The window of data may be reconstructed to form a 10 KHz frequency domain representation of the ROI. This high fidelity information can be used to calculate high fidelity values for parameters used to characterize varies types of tissue, such as benign and cancer. It should be understood that because the principles of the present invention utilize tones as ultrasound signals and that the SNR is high, that higher frequencies, such as 25 MHz-50 MHz, may be utilized. Such higher frequencies may result in higher resolution. In one embodiment, resolution settings may enable a technician to change resolution, which may change frequencies and/or frequency steps.

A Discrete Fourier Transform DFT can be computed using the Fast Fourier Transform FFT algorithm and in this case would be a 10 point DFT. The bank of FFTs and inverse FFTs may include a set of one-hundred, ten-point FFTs. The numbers of FFTs and number of points used vary depending on the length of the region of interest selected either by the operator or automatically by using a predetermined criterion, but a few default sets of values may be pre-selected to reduce the cognitive load on the operator. It should be understood that the number of points for the IFFT and FFT values may be changed if desired in the field due to the need to customize the ROI. In one embodiment, the ultrasound probe, as shown in FIG. 12, may include selectable buttons or other settings that may change the number of points being used for measurement.

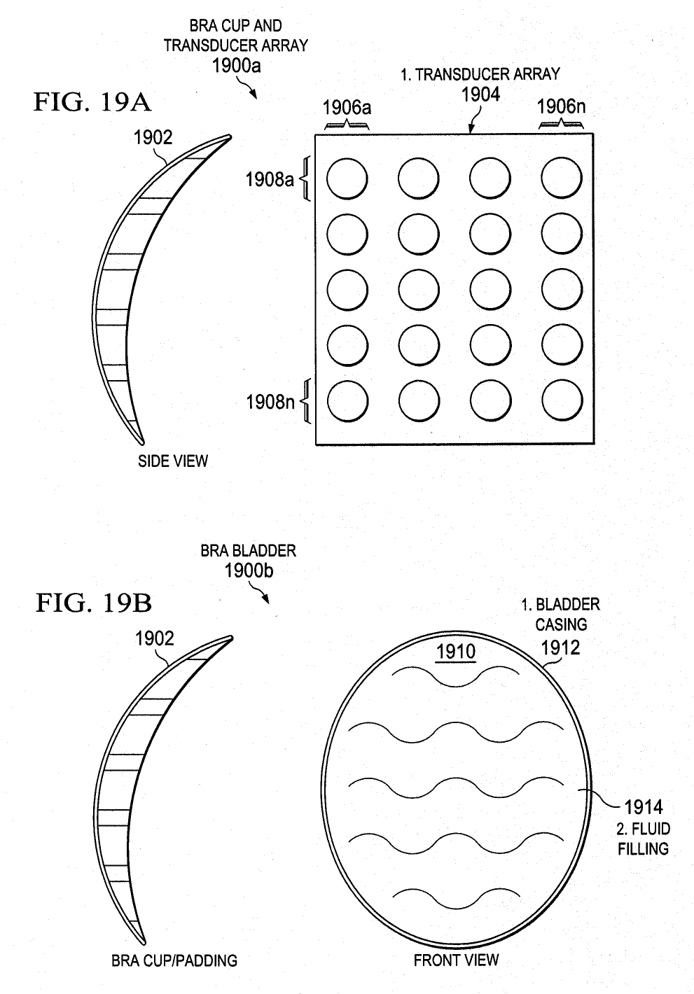

FIG. 19A is an illustration of components 1900a of an illustrative structure, in this case a bra, including a bra cup 1902 and an illustrative transducer array 1904 that may be used to perform ultrasound imaging in accordance with the principles of the present invention. In one embodiment, the bra cup 1902 may be formed of a stiff or semi-rigid material (e.g., plastic) to maintain a known geometric shape. For example, the known shape may be spherical. By maintaining a known shape, three dimensional images and precise measurement distances within tissue, such as a breast, may be more easily determined. The transducer array 1904 may be configured with columns 1906a-1906n and rows 1908a-1908n of transducers. The individual transducers may be in communication with individual couplers (not shown) and be capable of transmitting and receiving an ultrasound signal. Alternatively, the transducer array 1904 may be configured as having separate transducers for transmitting and receiving the incident and reflected ultrasound signals, respectively. The transducer array 1904 may have the individual transducers spaced at known distances apart from one another and in a particular pattern to enable the ultrasound system to have absolute and relative distance reference points from which to base measurements.

FIG. 19B is another illustration of components 1900b of the illustrative structure of FIG. 19A, in this case the “bra,” that utilizes an illustrative bladder 1910 that may be utilized to assist in performing ultrasound imaging in accordance with the principles of the present invention. The bladder 1910 may be configured with a bladder casing 1912 and fluid filling 1914. To minimize discontinuity reflections from the bladder 1910, the bladder casing 1912 and fluid filling 1914 may have an acoustic impedance approximately the same as water. Other tissue characteristic parameters may be matched to a known fluid (e.g., water), so that the ultrasound system may compensate or have substantially no impact as a result of communicating ultrasound signals through the bladder 1910 into tissue (or other object). Because anatomical regions, such as breasts, are different shapes and sizes of women, the bladder 1910 is used to substantially fill air gaps (i.e., air gaps that have minimal or no impact on an ultrasound measurement) that would otherwise exists and which would alter ultrasound measurements. As with conventional bras, fabric, straps, and fasteners may provide for an operator to position and maintain the transducer array 1904 on a patient. In one embodiment, the fabric on the cup of the bra, may include a pocket in which the cup 1902 and transducer array 1904 may be secured in position for use.

Although FIGS. 19A and 19B show a bra, it should be understood that many other structures may be utilized in accordance with the principles of the present invention to assist in measuring different anatomical regions. As an example, a helmet may be utilized to scan the scalp, where the helmet may be spherical, and a tube may be utilized to scan a finger, arm, or leg, where the tube may be cylindrical. Bladders may be configured to fill air gaps in the same or similar manner as bladder 1910 for the particular anatomical regions being scanned. In one embodiment, the bladder 1910 may be disposable, whereby the bladder casing 1912 and fluid filling 1914 may be biodegradable and not harmful to the environment. Alternatively, the bladder 1910 may be capable of being disinfected for re-use.

A two step process may be utilized for using the principles of the present invention, whereby a first step may scan a large number of positions on the breast using the bra cup 1902 and transducer array 1904 using the high accuracy, high resolution scanning processes previously described. A spherical enclosure, similar to a bra, may be used to hold a large number of transducers (e.g., transducer array 1904), which can be operated simultaneously. The bra component may save time for scanning, provide alignment accuracy for many scans, and provide comfort for the patient. A spherical enclosure, such as the bra cup 1902, may hold many small transducers in the form of the transducer array 1904. For example, a ½ inch Olympus ACCUSCAN-S A311S—can be focused from 0.75 inch to 8.40 inches. A semi-spherical container holds the transducers at ½ inch spacing. A semi-spherical bra cup having a 3-inch radius may contain about 100 square inches of material that may be arranged in a 10-square inch square pattern. In another embodiment, the transducer array 1904 may fill the container with 20×20=400 transducers.

The index matching bladder may be thick enough to provide a standoff. For example, for the 0.75 inches in the example transducer array 1904, the focus can start at the surface of the breast and continue several inches into the breast. Alternatively, a sheet of plastic piezoelectric material could be masked by a large number of apertures. The space between the breast and the container may be filled with a disposable bag of index matching fluid to fill substantially all air gaps between the breast and the transducer array 1904. The 400 transducers may be operated in parallel, where the data is stored in a memory buffer and read out over a bus sequentially using a multiplexer, for example. The 400 scans can be converted into a 3D model of the breast, where each scan might include a certain number of sample points, such as 500 points. Thus, the entire 3D model would include 20,000 voxels (volume elements). The high resolution image could be used to locate areas within the breast that should be imaged at a higher resolution. The second step may involve using the hand held ultrasound device disclosed hereinabove to examine more closely the volumes or regions identified by the multiple element devices.

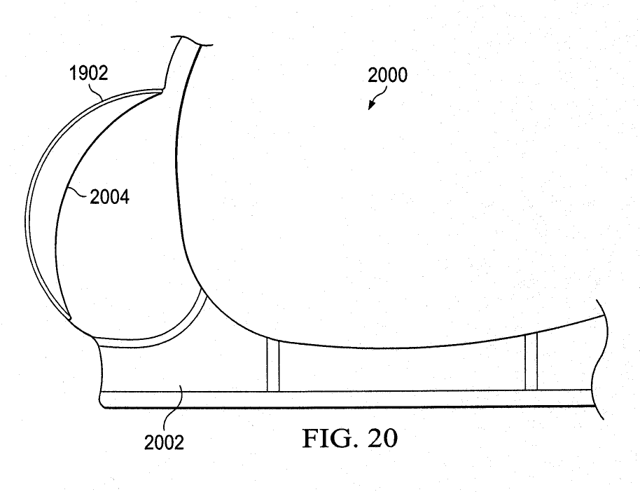

FIG. 20 is an illustration of an illustrative structure 2000, in this case a bra, that may be utilized to support and position a transducer array of FIG. 19A with respect to a patient. The structure 2000 may include the bra cup 1902 and a support band 2002 that includes fasteners (not shown), that may be utilized to secure the structure to a patient. The structure 2000 may further include a pocket 2004 in which the bra cup 1902 that is supporting the transducer array (not shown) may be positioned. As previously described, a bladder 1910 (FIG. 19B) may be inserted between the bra cup 1902 and breast of the patient to substantially eliminate air gaps, thereby improving ultrasound scans. And, because the bladder is disposable, sanitary conditions of the structure may be maintained.

The post-processing of the windowed frequency signal x(ω)ₒᵤₜ may be used to calculate or estimate one or more tissue characteristic parameters. As previously described, an attenuation or slope tissue characterization parameter (slope of ln(x(ω)) may be computed or estimated. Other tissue characteristic parameters, including phase velocity (speed of sound), impedance, and harmonic content of the tissue. From one or more of these computed or estimated parameters, a determination or estimate of the tissue type of the region of interest may be made. In one embodiment, the determination of the tissue type of the region of interest may be made using a neural network. Other mathematical and/or logic functions may be utilized in assisting in assessing the region of interest in accordance with the principles of the present invention. As an example, a neural network may be trained to identify different types of cancers based on one or more of the tissue characteristic parameters. The neural network may process the windowed frequency domain signal x( )ₒᵤₜ and/or the tissue characteristic parameters and determine which, if any, type of cancer the region of interest matches and, optionally, associate a probability with that assessment. In one embodiment, the ultrasound system may be configured to provide an output of cancer, no cancer, or unknown tissue type. Still yet, the ultrasound system may be configured to simply output cancer or no cancer for an operator. Such automated processing may be particularly helpful in regions of the world where medical training is limited.

With further regard to the tissue characterization parameters, and more specifically, the accuracy of the parameters used for ultrasound tissue characterization may include such quantities as: speed of sound in tissue V, ultrasound attenuation coefficient alpha (α). acoustic impedance Z, and harmonic frequency response divided by the fundamental response b/a. The acoustic impedance Z can also be used to help discriminate between lesions that are benign and cancer. The acoustic impedance is given by Z=DV, where D is the density of the medium and V is the velocity of sound in the tissue. The acoustic impedance can calculated from the reflected waveform R from the relationship Z=(1+R)/(1−R)Zo. Thus, because of the configuration of the ultrasound system utilizing the principles of the present invention, accurate measurement of the acoustic impedance Z may be achieved, As understood in the art, the velocity of sound V (alternatively listed as S or C hereinabove) and the attenuation alpha is usually expressed as a linear coefficient in dB/cm, such as 3 dB per centimeter.

One problem has long existed for pulse-based ultrasound devices has been the requirement for a large bandwidth in order to transmit and receive a narrow pulse. As understood in the art, the large bandwidth allows a large amount of noise into the system, which reduces the signal to noise ratio SNR of the measurement. The low SNR input signal creates a limit on the accuracy at which a downstream algorithm can be used to estimate the parameters of interest, such as α, Z and V.

The principles of the present invention use a very narrow bandwidth for each independent measurement using one tone at a time. The total bandwidth may be increased by stepping through many tones at different frequencies (e.g., 5 MHz->15 MHz). The larger effective bandwidth increases the spatial resolution, while the long duration tones increase the signal-to-noise ratio. The combined improvements allow for algorithms to estimate the features (α, Z, and V) accurately enough so that the algorithms can easily discriminate between malignant and benign tissue. The discrimination can be performed using any pattern recognition/machine intelligence for classification, such as neural networks, support vector machines, Hidden Markov Models, classification trees, etc.

Each of the tissue characterization parameters depend strongly on the accuracy of the frequency spectrum of the tissue that can be calculated from the ultrasound signal. The accuracy of the calculated or measured frequency spectrum depends on the frequency resolution and signal-to-noise of the spectrum. In conventional echo pulse ultrasound systems, the frequency resolution interval (i.e., smallest spacing between independent frequency samples) depends inversely on the pulse duration. Thus, both frequency resolution and SNR increase with pulse duration.

Longer pulse durations are desired for higher frequency resolution and higher SNR of the spectrum. However, longer durations limit the spatial resolution of echo pulse ultrasound systems, so in practice, the pulses of conventional echo pulse ultrasound systems are limited to about 1 μsec. For example, in conventional ultrasound imaging, a typical pulse is between two to four cycles of the center frequency. The center frequency of the pulse is usually from 3 MHz to 12 MHz, so the pulse duration varies from about 3×⅓ MHz=1 μsec to about 3× 1/12 MHz=0.25 μsec. Thus, the frequency resolution interval is about 1 to 4 MHz. The resolution of an FFT of the pulse has a resolution interval of between 1 to 4 MHz. and the SNR is limited since it is proportional to the duration of the pulse which is only about 1 μsec.

The frequency domain method used by the principles of the present invention transmits a tone for about 1 msec, which is 1,000 times longer in duration than the echo pulse systems. Thus, the SNR has a 10 Log(1,000)=30 dB improvement over echo pulse systems, and the frequency resolution is on the order of 1/1 msec=1 KHz. The improved frequency resolution and SNR of the Fourier spectrum (i.e., frequency spectrum) allows for much more accurate calculations of the tissue characterization parameters, which are usually calculated in the frequency domain. The high accuracy calculation of the parameters provides more reliable qualitative evaluation of the tissue types imaged by the ultrasound system.

The high accuracy calculations can be displayed separately or as overlays to the conventional ultrasound image. A conventional ultrasound image may be calculated simultaneously using the principles of the present invention by calculating A-scans using the inverse FFT on each frequency scan. The real-time simultaneous calculation of the A-scan with the tissue characterization parameter map allows for in-vivo as well as in-vitro use of the principles of the present invention.

The principles of the present invention reduce imaging noise as compared with conventional ultrasound systems. The instantaneous bandwidth is narrow, which reduces the noise bandwidth and, thus, noise is reduced in the measurements. The tradeoff is that scan-time is increased, but acceptable to an operator. Such a tradeoff can be seen in FIGS. 2 and 3, which show that the longer scan-time yields a narrower instantaneous bandwidth. However, as FIG. 3 shows, more scans are used to cover the same total bandwidth. The longer scan time is not a problem in breast or other scans since the ultrasound operator generally holds the transducer on each spot for a second or more, which is more than enough time to collect 1000 one millisecond scans.

High spatial resolution is required for early detection of breast cancer, where the main targets are the micro-calcifications that form in ducts and lobules. Micro-calcifications are on the order of 50 μm to 500 μm in size and are below the resolution limit of what conventional ultrasound systems can detect today. Utilizing the principles of the present invention, the resolution limit is extended for a low cost portable system by using longer scan times that provide more signal energy and less noise energy per measurement. By scanning multiple frequencies, large total bandwidths can be achieved. In addition, the total bandwidth is extended by use of rational models of the impedances in the frequency domain to increase the effective total bandwidth even further. The limits of the resolution may be extended so as to resolve structures less than 500 μm in size and frequently as low 50 μm. A first low resolution scan can be performed over the entire breast. The operator and the pattern recognition software can analyze the quick scan results to determine if high resolution scans are needed at any locations to determine whether micro-calcifications or stiff irregular lesions are present. Alternatively a large number of scans can be performed by using many transducers in a fixed geometric structure like a bra shape structure for breast scans.

The general problems with going to a higher frequency with wideband operation for ultrasound imaging systems include increased precision required of the analog front end electronics and the increased A/D conversion rates require expensive electronics. In digital ultrasound systems, the maximum imaging frequency is limited by the speed of the system's analog-to-digital converter. Conventional systems use A/D converters running at approximately 20 MHz. This limits the maximum imaging frequency to 10 MHz according to the Nyquist sampling theorem. The principles of the present invention provide for visualizing the reflection coefficient by using a narrow instantaneous bandwidth by sampling at baseband or an intermediate frequency (IF) low enough to reduce the sampling rate required as well as the cost of electronics. The down-conversion may be performed in either the frequency or time domain. The resulting electronics and analog-to-digital (A/D) converter becomes comparable to audio electronics, which are inexpensive. The trade-off in using such an inexpensive architecture is the scan time for the instantaneous bandwidth. In order to scan a total broad bandwidth of N times the narrow instantaneous bandwidth, an increase of N for scan time is used. The principles of the present invention may use a two transducer probe, such as the Olympus Panametrics NDT DHC713-RM probe, which can both transmit and receive signals at the same time by using two transducers in the probe simultaneously.

A 1 msec scan would travel about 0.75 meters round trip into the body. If a total bandwidth of 10 MHz is used with an instantaneous bandwidth of 10 KHz, then N=1000 and the total time at each site on the patient's body (e.g., breast) uses 1 second to scan. In another embodiment, the instantaneous bandwidth could be 100 KHz and each site would take one tenth of a second to scan. Software algorithms may include the use of vector fitting or similar pole fitting algorithms to model the acoustic impedance as a rational function of frequency. The models obtained by these methods may be used to extrapolate the frequency range so as to be larger than the range actually scanned. The larger bandwidth scanned and extrapolated in the frequency domain provides increased spatial resolution to provide for better visualization of details, such as small micro-calcifications. The use of vector fitting algorithms can reduce the scan time by a factor of 20-90%. The low instantaneous bandwidth and lower IF frequency used greatly reduces the cost of the components to reconstruct high fidelity images similar to those provided by much more costly machines. The use of spectral extrapolation algorithms also reduces scan time.

In the USA, most women are have breast scans using mammography on a regular basis and tumors are usually found and treated early. However, in many other parts of the world women do not get regular screening and can come to point of care unit with a unchecked tumor. As a result of providing a low cost, portable point of care unit utilizing the principles of the present invention diagnoses of breast cancer may be provided to women in impoverished nations. The ability to determine cancer versus benign tissue is also enhanced. The enhance contrast resolution of the acoustic impedance is utilized to determine if a lesion is cancer.

An operator using an ultrasound system that incorporates the principles of the present invention sees a very “clean” (i.e., minimal speckle) high-fidelity image with much better spatial resolution than conventional ultrasound systems. The operator may see smaller objects than seen previously, and the image is much less mottle due to the reduced effects of speckle noise. Furthermore, subtle variations in tissue may be seen to more easily identify a tumor or other masses. Since cancer is much more jagged-shaped and much harder tumors, and the benign lesions of fat and/or blood, etc., are usually more spherical, more smooth, more regular, and softer, the principles of the present invention allow for easier identification of cancer cells and other tissue features.

The resolution of the system utilizing the principles of the present invention are improved over conventional ultrasound because the speed of the electronics are slower, one frequency is sampled at a time, which also results in the electronics being much, much more inexpensive (e.g., a low speed, low cost A/D converter may be used). The bandwidth is very narrow so all the analog electronics are less expensive. Performance of the principles of the present invention is mainly achieved through the use of sophisticated software, where the algorithms compensate for the low cost electronics. Scan times which are on the order of tenths of seconds or a second in the present invention rather than microseconds as in conventional ultrasound imaging systems.

Although described as an ultrasound system that is conventionally used to image parts of human or animal bodies, the principles of the present invention provide for other uses. If being used for other purposes, such as imaging below ground, different frequencies and/or power levels may be utilized. The same or similar principles of measuring in the frequency domain, converting to the time domain to identify changes in the medium being imaged, and converting back to the frequency domain to determine characterization parameters for use in determining a type of material being imaged.

### Select Features

A new device and method comprising a high precision measurement of acoustic impedance of a tumor, thereby distinguishing between the classes of malignant and benign tumors as well as identifying the specific types of tissue within each class.

The device may use a high bandwidth ultrasound transducer (for example 7.5-12 MHz) connected to a directional coupler or multiple transducers and then to a continuous wave signal generator, which is stepped through various frequencies and is used to measure the reflection coefficient for each of the frequencies scanned within the wide band.

The reflection coefficient may be collected as a function of frequency and inverse Fourier transformed into the spatial domain in order to isolate the individual tumors. Each region of interest may then filtered out of the scan.

Each region of interest may then be transferred back to the frequency domain and the acoustic impedance as a function of frequency may be obtained. A parametric representation may then formed of the tumors' acoustic impedance, speed of sound, attenuation and/or harmonic content as a function of frequency.

The high-fidelity calculations of local tissue attenuation and speed of sound can be used to correct or compensate the A-scan before display so as to enhance the fine details for better diagnosis capability.

The parameters may then used in a standard pattern recognition classifier, such as a neural network, K-Means classifier, Support Vector Machine, etc., to identify the tumor and which class of cancerous or noncancerous tissue it belongs.

The previous description is of a preferred embodiment for implementing the invention, and the scope of the invention should not necessarily be limited by this description. The scope of the present invention is instead defined by the following claims.

What is claimed:

1. An ultrasound system, comprising:
a processing unit;
a transducer;
a signal generator in communication with said computing device and said transducer; and
a receiver in communication with said processing unit and said transducer, said computing device configured to cause said signal generator to generate a set of ultrasound signals ranging from a first frequency to a second frequency and output the set of ultrasound signals via said transducer into an object, said receiver being configured to receive a set of reflected ultrasound signals via said transducer, said processing unit further being configured to:
calculate a set of reflection signals that are integrated over a dwell time, the set of reflection signals being stored as a function of frequency;
subsample the set of reflection signals;
convert the subsampled set of reflection signals into the time domain;
identify a region of interest in the object based on the subsampled set of reflection signals in the time domain;
convert the subsampled set of reflection signals from the time domain into the frequency domain;
combine, in the frequency domain, the converted set of subsampled reflection signals;
determine at least one characteristic parameter associated with the identified region of interest in the frequency domain; and
output information based on the determined at least one characteristic parameter to a user of the ultrasound system.

2. The ultrasound system according to claim 1, wherein said processing unit is further configured to extrapolate each individual reflection signal in the frequency domain to obtain an effective wider bandwidth than the bandwidth ranging from the first to the second frequency.

3. The ultrasound system according to claim 1, where said processing unit, in subsampling, is configured to demultiplex the set of reflection signals, and in combining, is configured to multiplex the converted set of subsampled reflection signals, and wherein the demultiplex and multiplex operations have one-to-one correspondence.

4. The ultrasound system according to claim 1, wherein the dwell time T_{dwell} is longer than the time for sound to travel the length of the region of interest.

5. The ultrasound system according to claim 1, wherein said computing system further is configured to determine whether the identified region of interest includes cancerous or pathologic tissue based on the at least one characteristic parameter using pattern recognition techniques.

6. The ultrasound system according to claim 5, wherein said computing system is further configured to display the at least characteristic parameter, enable a user to indicate whether the at least one characteristic parameter is representative of cancer, and store the indication in association with the at least one characteristic parameter for learning purposes.

7. The ultrasound system according to claim 1, wherein the set of ultrasound signals are stepped from the first frequency to the second frequency at a step frequency spacing, said receiver being further configured to receive each of the respective ultrasound signals and harmonics of the respective ultrasound signals, said processing unit further being configured to process the respective ultrasound signals and the harmonics of the respective ultrasound signals in determining the at least one characteristic parameter.

8. The ultrasound system according to claim 1, wherein said processing unit is further configured to utilize a neural network to process the at least one characteristic parameter to determine a substance of the identified region of interest.

9. The ultrasound system according to claim 1, wherein said processing unit, in outputting information, is configured to indicate whether a particular substance is identified in the identified region of interest.

10. The ultrasound system according to claim 9, wherein the particular substance is cancerous tissue.

11. The ultrasound system according to claim 1, wherein said processing unit, in identifying a region of interest, is further configured to define a window around the region of interest, and wherein said processing unit is further configured to convert a windowed portion of the demultiplexed set of reflection signals from the time domain into the frequency domain.

12. The ultrasound system according to claim 1, wherein said processing unit, in outputting the information includes outputting an A-scan compensated by the at least one characteristic parameter, the at least one characteristic parameter including at least one of local tissue attenuation and speed of sound through the region of interest.

13. The ultrasound system according to claim 1, wherein said processing unit, in outputting the information includes outputting an A-scan along with a highlight of the identified region of interest on the A-scan.

14. The ultrasound system according to claim 1, further comprising a directional coupler in communication with said transducer, and wherein said transducer includes one and only one transducer element.

15. The ultrasound system according to claim 1, further comprising a support structure having a known geometric configuration, said support structure being configured to support a grid of transducers.

16. A method for performing an ultrasound, said method comprising:
generating a set of continuous tone signals in the frequency domain for injection into an object;
receiving a corresponding set of reflected tone signals in the frequency domain;
converting the set of reflected tone signals from the frequency domain to the time domain to create a set of time domain signals;
identifying at least one region of interest from the set of time domain signals;
defining a window around the identified region of interest in the set of time domain signals;
converting the windowed time domain signals from the time domain to the frequency domain to create a set of windowed frequency domain signals;
calculating at least one characteristic parameter from the set of windowed frequency domain signals; and
outputting information based on the calculate at least one characteristic parameter to a user of the ultrasound system.

17. The method according to claim 16, further comprising determining a probability based on the at least one characteristic parameter that the region of interest includes a particular substance.

18. The method according to claim 17, wherein determining includes determining a probability that the particular substance is cancer.

19. The method according to claim 16, wherein generating a set of continuous tone signals includes stepping through a continuous set of tone signals at different frequencies, each tone signal being generated for at least approximately 1 ms.

20. The method according to claim 16, further comprising performing a matched filter over a dwell time T_{dwell} on the reflected tone signals to generate reflection coefficients.

21. The method according to claim 20, further comprising:
demultiplexing the set of reflected tone signals; and
multiplexing the set of windowed frequency domain signals.

22. The method according to claim 21, further displaying the multiplexed set of windowed frequency domain signals.

23. The method according to claim 22, wherein displaying the multiplexed set of windowed frequency domain signals includes displaying the multiplexed set of windowed frequency domain signals on an A-scan.

24. The method according to claim 23, wherein further comprising compensating the A-scan by the at least one characteristic parameter, the at least one characteristic parameter including at least one of local tissue attenuation and speed of sound through the region of interest.

25. The method according to claim 16, wherein determining a probability includes using a neural network to determine the probability.

26. The method according to claim 16, wherein injecting and receiving the respective tone signals includes injecting and receiving the respective tone signals via a single transmit and receive device.

27. The method according to claim 16, wherein converting the set of reflected tone signals includes converting the set of reflected tone signals including at least one harmonic frequency associated with each tone, and wherein identifying at least one region of interest includes using the at least one harmonic frequency associated with each tone.

28. The method according to claim 16, wherein injecting the set of continuous tone signals includes injecting the set of continuous tone signals that includes a tone that exceeds 25 MHz, and wherein receiving corresponding reflected tone signals includes receiving corresponding reflected tone signals over a dwell time T_{dwell} of at least approximately 1 ms.

29. The method according to claim 16, wherein calculating includes calculating an amount of non-linearity a harmonic of each of the set of windowed frequency domain signals.

30. The method according to claim 16, further comprising interpolating and extrapolating frequency information for each set of windowed frequency domain signals to increase spatial and contrast resolution.

31. The method according to claim 16, further comprising:
transmitting a plurality of sets of continuous tone signals from predetermined, fixed positions into the object; and
receiving the plurality of sets of reflected tone signals from predetermined, fixed positions.

32. The method according to claim 31, wherein transmitting includes transmitting the plurality of sets of continuous tone signals through a disposable bladder that (i) substantially fills air gaps between transducers configured to transmit and the object and (ii) has approximately the same acoustic impedance as water.

33. A method for performing an ultrasound, said method comprising:
generating a set of continuous tone signals in the frequency domain for injection into an object;
receiving a corresponding set of reflected tone signals in the frequency domain;
converting the set of reflected tone signals from the frequency domain to the time domain to create a set of time domain signals;
identifying at least one region of interest from the set of time domain signals;
converting the time domain signals from the time domain to the frequency domain to create a set of frequency domain signals;
calculating at least one characteristic parameter from the set of frequency domain signals;
compensating the set of frequency domain signals by the at least one characteristic parameter to create a compensated set of frequency domain signals; and
displaying the compensated set of frequency domain signals.

34. The method according to claim 33, wherein the at least one characteristic parameter includes attenuation.

36. The method according to claim 33, wherein displaying includes displaying an A-scan with the compensated set of frequency domain signals.

37. The method according to claim 33, further comprising down-converting the set of reflected tone signals.

38. The method according to claim 37, further comprising performing a matched filter on the down-converted set of reflected tone signals.

39. A structure for supporting an ultrasound probe for imaging an anatomical region of a patient, said structure comprising:
a first member being semi-rigid and having a pre-determined geometrical shape;
an array of transducers supported by and positioned relative to said first member;
a bladder having a size and shape that conforms to being positioned between said first member and the anatomical region of the patient; and
a securing member that causes said first member, array of transducers, and bladder to maintain position relative to the anatomical region of the patient.

40. The structure according to claim 39, wherein said first member is semi-hemispherical.

41. The structure according to claim 39, wherein said securing member is a strap in the shape of a bra, and wherein the anatomical region is a breast.
